(19) United States
(12) Patent Application Publication
(10) Pub. No.: US 2026/0347481 A1
(43) Pub. Date: Nov. 5, 2026

(54) ANTI-MEEGFR ANTIBODY, ANTIGEN-BINDING FRAGMENT THEREOF, AND USE THEREOF

(71) Applicant: Mien-chie Hung, Taichung City (TW)

(72) Inventors: Mien-chie Hung, Taichung City (TW); Shao-chun Wang, Taichung City (TW); Hirohito Yamaguchi, Taichung City (TW); Jung-mao Hsu, Taichung City (TW); Yi-chun Shen, Taichung City (TW); Hong-wei Li, Taichung City (TW); You-zhe Lin, Taichung City (TW)

(73) Assignee: Mien-chie Hung, Taichung City (TW)

(21) Appl. No.: 19/106,441

(22) PCT Filed: Aug. 30, 2023
(86) PCT No.: PCT/CN2023/115936

§ 371 Date: Feb. 25, 2025

Related U.S. Application Data
(60) Provisional application No. 63/402,545, filed on Aug. 31, 2022.

(51) Int. Cl. C07K 16/28; A61K 31/513; A61K 31/7068; A61K 39/395; A61K 40/15; A61K 40/31; A61K 40/42; A61K 47/68; A61P 35/00; C12N 5/0783; G01N 33/57515

(52) U.S. Cl. CPC C07K 16/2863; A61K 31/513; A61K 31/7068; A61K 39/3955; A61K 40/15; A61K 40/31; A61K 40/4204; A61K 47/68031; A61K 47/6849; A61P 35/00; C12N 5/0646; G01N 33/57515; C07K 2317/53; C07K 2317/565; C07K 2317/622; C07K 2317/92; C07K 2319/02; C07K 2319/03; C12N 2510/00; G01N 2333/71

(57) ABSTRACT

An anti-methylated epidermal growth factor receptor (meEGFR) antibody or an antigen-binding fragment thereof, a detection kit, a method for detecting a meEGFR positive cancer, a pharmaceutical composition for treating a cancer, a meEGFR-specific chimeric antigen receptor, a nucleic acid encoding the meEGFR-specific chimeric antigen receptor, a meEGFR-specific chimeric antigen receptor expressing cell, an innate cell engager, and an antibody conjugate are provided. The anti-meEGFR antibody or the antigen-binding fragment thereof specifically binds to the meEGFR, and the meEGFR has asymmetric dimethylation modification at R198 and R200 residues.

### BACKGROUND

### Technical Field

The present disclosure relates to an anti-epidermal growth factor receptor (EGFR) antibody or an antigen-binding fragment thereof and uses thereof. More particularly, the present disclosure relates to an anti-methylated epidermal growth factor receptor (meEGFR) antibody or an antigen-binding fragment thereof and uses thereof.

### Description of Related Art

Cancer, also known as malignant tumor, is a disease caused by abnormal cell proliferation, where these proliferating cells may invade other parts of the body due to a malfunction in the mechanism controlling cell division and proliferation. The number of people suffering from cancer worldwide has a growing trend, and cancer has been ranked first among the top ten causes of death for twenty-seven consecutive years. Cancer treatment mainly includes radiotherapy, chemotherapy, targeted therapy, and surgery. Despite advancements in drugs and surgical techniques in recent years, the five-year survival rate for advanced patients remains quite low, highlighting the importance of developing a new treatment strategy.

The epidermal growth factor receptor (EGFR) is a type of receptor tyrosine kinase, and mutations or overexpression of EGFR can be observed in various types of cancer, which are related to tumor proliferation, angiogenesis, and metastasis. Currently, EGFR-targeted therapies have been developed and applied clinically, including the EGFR-tyrosine kinase inhibitor (TKI) such as gefitinib and erlotinib, and the monoclonal antibody such as cetuximab and panitumumab. In the triple negative breast cancer (TNBC) tumor, the overexpression rate of EGFR is as high as 70%, which is much higher than other molecular subtypes of breast cancer. High EGFR expression is also significantly associated with poorer progression-free survival and overall survival in TNBC. Therefore, EGFR is a potential therapeutic target for TNBC and advanced breast cancer.

Regarding EGFR, several EGFR-TKIs have been approved so far, but secondary drug resistance mutations are a critical issue in the treatment process. The first three generations of TKIs all target the intracellular ATP-binding site and inhibit EGFR activity by blocking phosphorylation thereof. However, to date, many clinical trials evaluating the efficacy of EGFR-targeted drugs in treating TNBC patients have failed to show or confirm their therapeutic benefits. Therefore, a new treatment strategy needs to be developed to replace conventional EGFR-targeted drugs. Post-translational modifications are important mechanisms for regulating protein physiological functions. However, there are currently no antibody drugs targeting specific protein post-translational modifications in clinical treatment and on the market. Targeting other post-translational modification pathway of EGFR is a potential direction for improving drug resistance of EGFR-TKI.

### SUMMARY

According to one aspect of the present disclosure is to provide an anti-meEGFR antibody or an antigen-binding fragment thereof, which specifically binds to a methylated epidermal growth factor receptor (meEGFR) having asymmetric dimethylation modifications at R198 and R200 residues.

The anti-meEGFR antibody or the antigen-binding fragment thereof includes a heavy chain variable region (V_{H}) and a light chain variable region (V_{L}), wherein the V_{H} includes a heavy chain complementarity-determining region 1 (HCDR1), a heavy chain complementarity-determining region 2 (HCDR2), and a heavy chain complementarity-determining region 3 (HCDR3), and the V_{L} includes a light chain complementarity-determining region 1 (LCDR1), a light chain complementarity-determining region 2 (LCDR2), and a light chain complementarity-determining region 3 (LCDR3). The sequences of the HCDR1, the HCDR2, the HCDR3, the LCDR1, the LCDR2, and the LCDR3 are selected from the group consisting of: the HCDR1 of SEQ ID NO: 7, the HCDR2 of SEQ ID NO: 8, the HCDR3 of SEQ ID NO: 9, the LCDR1 of SEQ ID NO: 10, the LCDR2 of SEQ ID NO: 11, and the LCDR3 of SEQ ID NO: 12; the HCDR1 of SEQ ID NO: 13, the HCDR2 of SEQ ID NO: 14, the HCDR3 of SEQ ID NO: 15, the LCDR1 of SEQ ID NO: 16, the LCDR2 of SEQ ID NO: 17, and the LCDR3 of SEQ ID NO: 18; and the HCDR1 of SEQ ID NO: 19, the HCDR2 of SEQ ID NO: 20, the HCDR3 of SEQ ID NO: 21, the LCDR1 of SEQ ID NO: 22, the LCDR2 of SEQ ID NO: 23, and the LCDR3 of SEQ ID NO: 24.

According to another aspect of the present disclosure is to provide a detection kit for detecting a meEGFR-positive cancer. The detection kit includes the anti-meEGFR antibody or the antigen-binding fragment thereof according to the foregoing aspect.

According to still another aspect of the present disclosure is to provide a method for detecting a meEGFR-positive cancer, which includes the following steps: providing a test sample, providing the detection kit according to the foregoing aspect, performing a binding step, and performing a detection step. In the binding step, the test sample is contacted with the anti-meEGFR antibody or the antigen-binding fragment thereof to form a binding reaction. The detection step is to detect whether the test sample includes an antibody-cancer cell complex.

According to yet another aspect of the present disclosure is to provide a pharmaceutical composition for treating a cancer including the anti-meEGFR antibody or the antigen-binding fragment thereof according to the foregoing aspect and a pharmaceutically acceptable carrier.

According to still another aspect of the present disclosure is to provide a meEGFR-specific chimeric antigen receptor including an extracellular domain and an intracellular signaling domain. The extracellular domain is for recognizing a methylated epidermal growth factor receptor (meEGFR), and the meEGFR has asymmetric dimethylation modifications at R198 and R200 residues, and the extracellular domain includes the anti-meEGFR antibody or the antigen-binding fragment thereof according to the foregoing aspect.

According to yet another aspect of the present disclosure is to provide a nucleic acid encoding the meEGFR-specific chimeric antigen receptor according to the foregoing aspect.

According to still another aspect of the present disclosure is to provide a meEGFR-specific chimeric antigen receptor expressing cell including an immune cell and the nucleic acid according to the foregoing aspect, wherein the meEGFR-specific chimeric antigen receptor expressing cell is obtained by transfecting the nucleic acid into the immune cell.

According to yet another aspect of the present disclosure is to provide a pharmaceutical composition for treating a cancer including the meEGFR-specific chimeric antigen receptor expressing cell according to the foregoing aspect and a pharmaceutically acceptable carrier.

According to still another aspect of the present disclosure is to provide an innate cell engager including a meEGFR antigen recognition domain and at least one immune cell receptor binding domain, wherein the meEGFR antigen recognition domain includes the anti-meEGFR antibody or the antigen-binding fragment thereof according to the foregoing aspect, and the at least one immune cell receptor binding domain has binding specificity for one or more of CD3, CD8, CD16, CD19, or NKG2D.

According to yet another aspect of the present disclosure is to provide an antibody conjugate including the anti-meEGFR antibody or the antigen-binding fragment thereof according to the foregoing aspect and an effector molecule, wherein the effector molecule is conjugated with the anti-meEGFR antibody or the antigen-binding fragment thereof through a chemical bond or a linker, and the effector molecule is a toxin, a growth inhibitor, a toxic protein, a radionuclide, a radioactive isotope, a chemotherapeutic drug, or a combination thereof.

### BRIEF DESCRIPTION OF THE DRAWINGS

The present disclosure can be more fully understood by reading the following detailed description of the embodiment, with reference made to the accompanying drawings as follows:

FIG. 1 is a schematic diagram of the phage display technology for screening an anti-meEGFR antibody or an antigen-binding fragment thereof according to one aspect of the present disclosure;

FIG. 2A and FIG. 2B show the sodium dodecyl sulfate polyacrylamide gel electrophoresis (SDS-PAGE) analysis results of the anti-meEGFR antibody or the antigen-binding fragment thereof according to one aspect of the present disclosure;

FIG. 3A, FIG. 3B, and FIG. 3C show the binding affinity analysis results of the anti-meEGFR antibody or the antigen-binding fragment thereof with a target peptide according to one embodiment of the present disclosure;

FIG. 4 is a step flow chart of a method for detecting a meEGFR-positive cancer according to another aspect of the present disclosure;

FIG. 5A and FIG. 5B show the binding specificity analysis results of a detection kit according to still another aspect of the present disclosure;

FIG. 6A, FIG. 6B, and FIG. 6C show the analysis results of the meEGFR expression in breast cancer patients;

FIG. 6D shows the overall survival analysis results of the meEGFR-positive patients and the meEGFR-negative breast cancer patients;

FIG. 6E shows the disease-free survival analysis results of the meEGFR-positive patients and the meEGFR-negative breast cancer patients;

FIG. 7A, FIG. 7B, FIG. 7C, FIG. 7D, FIG. 7E, and FIG. 7F show the analysis results of tumor cell death induced by a pharmaceutical composition for treating a cancer according to one embodiment of yet another aspect of the present disclosure;

FIG. 8A and FIG. 8B show the analysis results of tumor cell death induced by the pharmaceutical composition for treating the cancer according to yet another embodiment of another aspect of the present disclosure;

FIG. 9 is a theoretical structural diagram of a meEGFR-specific chimeric antigen receptor according to still another aspect of the present disclosure;

FIG. 10A and FIG. 10B show the analysis results of specific lysis of tumor cells by a meEGFR-specific chimeric antigen receptor expressing cell according to still another aspect of the present disclosure;

FIG. 11A and FIG. 11B show the analysis results of an antibody conjugate being internalized by tumor cells according to one embodiment of yet another aspect of the present disclosure; and

FIG. 12 shows the analysis results of tumor cell death induced by the antibody conjugate according to another embodiment of yet another aspect of the present disclosure.

### DETAILED DESCRIPTION

Unless otherwise noted, all terms, symbols or other scientific terms or terms used in the present disclosure have the meanings that are commonly understood by person having ordinary skill in the art. In some cases, terms with conventional meanings are defined herein for clarity and/or immediate reference, and the definitions incorporated herein should be construed as not necessarily substantial different from the conventional meanings in the art. Many of the techniques and procedures described or referenced herein are well known and routinely used by those skilled in the art. Where appropriate, unless otherwise stated, procedures for the use of commercially available kits and reagents are generally performed according to instructions and/or parameters defined by the manufacturer.

The extracellular structural region of epidermal growth factor receptor (EGFR) includes two specific arginine (R198 and R200) residues that can be methylated by protein arginine methyltransferase 1 (PRMT1). The term “methylated epidermal growth factor receptor (meEGFR)” described in the specification refers to EGFR with asymmetric dimethylation modifications at the R198 and R200 residues in the extracellular domain. The asymmetric methylation modification of R198 and R200 results in the sustained activation of the meEGFR, which accelerates cancer cell proliferation and induces resistance to various EGFR inhibitors. For example, some cancer patients undergoing clinical trials with the FDA-approved anti-EGFR antibody cetuximab have experienced treatment failure and developed resistance to cetuximab.

The term “antibody” described in the specification refers to an immunoglobulin, which is composed of two identical heavy chains and two identical light chains linked by interchain disulfide bonds to form a tetrapeptide structure. The amino acid composition and arrangement order of the constant region (C_{H}) of the heavy chain of different immunoglobulins are different, so their antigenicity is also different. Accordingly, the immunoglobulins can be classified into five classes, also known as isotypes: IgM, IgD, IgG, IgA, and IgE, with corresponding heavy chains being μ chain, δ chain, γ chain, α chain, and ε chain, respectively. The same class of Ig can be further classified into different subclasses based on the amino acid composition of the hinge region and the number and position of disulfide bonds in the heavy chain. For example, IgG can be classified into IgG1, IgG2, IgG3, and IgG4. The light chains are classified into κ chain or λ chain based on differences in the constant region. Each class of Ig can have either κ chain or λ chain.

The anti-meEGFR antibody or the antigen-binding fragment thereof of the present disclosure includes a “functional portion”, and the functional portion refers to a portion that shares at least one characteristic, not necessarily the same amount, with the antibody. The functional portion can bind to the same antigen, though with varying degrees of binding. The functional portion typically includes at least one heavy chain variable region (V_{H}) and one light chain variable region (V_{L}). In some cases, the functional portion can only include one V_{H}.

The anti-meEGFR antibody or the antigen-binding fragment thereof of the present disclosure can be a human antibody, wherein the V_{H} can further include a C_{H}, and the C_{H} includes human IgG1, IgG2, IgG3, IgG4, or variants thereof. The V_{L} can further include a light chain constant region (C_{L}), and the C_{L} includes human κ chain, λ chain, or variants thereof.

The anti-meEGFR antibody of the present disclosure can include a murine antibody, a chimeric antibody, a humanized antibody, preferably the humanized antibody.

The term “murine antibody” in the present disclosure refers to a monoclonal antibody specifically targeting human meEGFR, prepared using knowledge and skills in the field. In the preparation, the meEGFR is used as an antigen to immunize test subjects, followed by isolating hybridomas that express antibodies with the desired sequence or functional characteristics. In a preferred embodiment of the present disclosure, the murine anti-meEGFR antibody can further include murine κ chain, λ chain, or variants thereof as the C_{L}, or can further include murine IgG1, IgG2, IgG3, or variants thereof as the C_{H}.

The term “chimeric antibody” refers to an antibody formed by fusing the variable region of a murine antibody with the constant region of a human antibody, which can reduce the immune response induced by the murine antibody. To construct a chimeric antibody, a hybridoma secreting murine-specific monoclonal antibodies is first established, and the variable region gene of the murine antibody is then cloned. Next, the constant region gene of the human antibody is cloned as needed, and the murine variable region gene is fused with the human constant region gene to form a chimeric gene, which is then inserted into a human vector. The chimeric antibody is ultimately expressed in eukaryotic system or prokaryotic system. In a preferred embodiment of the present disclosure, the light chain of the chimeric antibody further includes the human κ chain or λ chain or variants thereof as the C_{L}. The heavy chain of the chimeric antibody further includes the human IgG1, IgG2, IgG3, IgG4, or variants thereof as the C_{H}. The constant region of the human antibody can be selected from human IgG1, IgG2, IgG3, or IgG4 or variants thereof as the C_{H}, preferably, including human IgG2 or IgG4 as the C_{H}.

The term “humanized antibody,” also known as CDR-grafted antibody, refers to an antibody produced by grafting the complementary determining region (CDR) sequences of a murine antibody into the variable region framework of a human antibody, i.e., into different human antibody isotype frameworks. This approach can overcome the strong immune response induced by the chimeric antibody due to carrying a large amount of murine protein components. Such framework sequences can be obtained from public DNA databases or published references including species' antibody gene sequences. To avoid a decrease in activity caused by a decrease in immunogenicity of the humanized antibody, a minimum of reverse mutation or back mutation can be performed on the framework sequence of the variable region of the humanized antibody to maintain activity. The humanized antibody of the present disclosure also includes the humanized antibody that further undergoes affinity maturation of CDR obtained through phage display technology.

The term “polymeric antibody” refers to the anti-meEGFR antibody including more than two basic units, which can be a dimer, trimer, or tetramer.

The term “antigen-binding fragment” described in the present disclosure refers to a Fab fragment, a Fab′ fragment, or a F(ab′)2 fragment with antigen-binding activity, as well as a fragment variable (Fv), a single-chain variable fragment (scFv) that can bind to the meEGFR; which includes one or more CDRs selected from SEQ ID NO: 7 to SEQ ID NO: 24 of the anti-meEGFR antibody or the antigen-binding fragment thereof of the present disclosure.

The term “Fab fragment” refers to a fragment composed of a light chain and a heavy chain, consisting of the C_{H}1 and the variable region. The heavy chain of the Fab fragment cannot form disulfide bonds with another heavy chain molecule.

The term “Fab′ fragment” refers to a fragment including a light chain and a portion of a heavy chain that includes the V_{H} domain, the C_{H}1 domain, and the region between the C_{H}1 domain and the C_{H}2 domain, thereby forming interchain disulfide bonds between the two heavy chains of two Fab′ fragments to form an F(ab′)2 fragment.

The term “F(ab′)2 fragment” refers to a fragment including two light chains and two heavy chains, wherein the two heavy chains include the constant region between the C_{H}1 domain and the C_{H}2 domain, thereby forming interchain disulfide bonds between the two heavy chains. Therefore, the F(ab′)2 fragment is composed of two Fab′ fragments held together by disulfide bonds between the two heavy chains.

The term “fragment variable (Fv)” refers to a smallest antibody fragment including the V_{H} and the V_{L} of the antibody but without the constant region and having all antigen-binding sites. Generally, the Fv also includes a polypeptide linker between the V_{H} domain and the V_{L} domain and can form the structure required for antigen binding. The two antibody variable regions can also be linked into a single polypeptide chain using different linkers, resulting in a single-chain variable fragment (scFv) or a single-chain Fv (sFv).

The term “single domain antibody (sdAb)” refers to a type of antibody that lacks the light chain and consists only of the V_{H}. A complete antibody includes two immunoglobulin light chains and two heavy chains, with a molecular weight of approximately 150-160 kDa. In contrast, the molecular weight of the single domain antibody is only about 12-15 kDa. Due to the small molecular weight of the single domain antibody, it is also known as a nanobody. Despite its simple structure, the single domain antibody can achieve antigen-binding affinity comparable to or even higher than that of a complete antibody.

The term “binding to meEGFR” refers to the ability to interact with human meEGFR. The term “antigen-binding site” refers to a discontinuous three-dimensional spatial site on the antigen recognized by the anti-meEGFR antibody or the antigen-binding fragment thereof of the present disclosure.

The term “pharmaceutical composition” described in the specification refers to a mixture including one or more anti-meEGFR antibodies or antigen-binding fragments thereof described herein and a pharmaceutically acceptable carrier. Examples of the pharmaceutically acceptable carrier include physiologically/pharmaceutically acceptable carriers and excipients. The purpose of the pharmaceutical composition is to facilitate the administration to a biological organism and promote the absorption of the active ingredient to exert biological activity.

The following specific examples are used to further illustrate the present disclosure, in order to benefit the person having ordinary skill in the art, and can fully utilize and practice the present disclosure without excessive interpretation. These examples should not be regarded as limiting the scope of the present disclosure, but is used to illustrate how to implement the materials and methods of the present disclosure.

### I. An Anti-meEGFR Antibody or an Antigen-Binding Fragment Thereof

An anti-meEGFR antibody or an antigen-binding fragment thereof of the present disclosure specifically binds to meEGFR, and the meEGFR has asymmetric dimethylation modifications at R198 and R200 residues. The anti-meEGFR antibody or the antigen-binding fragment thereof includes a heavy chain variable region (V_{H}) and a light chain variable region (V_{L}), wherein the V_{H} includes a heavy chain complementarity-determining region 1 (HCDR1), a heavy chain complementarity-determining region 2 (HCDR2), and a heavy chain complementarity-determining region 3 (HCDR3), and the V_{L} includes a light chain complementarity-determining region 1 (LCDR1), a light chain complementarity-determining region 2 (LCDR2), and a light chain complementarity-determining region 3 (LCDR3). The sequences of the HCDR1, the HCDR2, the HCDR3, the LCDR1, the LCDR2, and the LCDR3 are selected from the group consisting of: the HCDR1 of SEQ ID NO: 7, the HCDR2 of SEQ ID NO: 8, the HCDR3 of SEQ ID NO: 9, the LCDR1 of SEQ ID NO: 10, the LCDR2 of SEQ ID NO: 11, and the LCDR3 of SEQ ID NO: 12; the HCDR1 of SEQ ID NO: 13, the HCDR2 of SEQ ID NO: 14, the HCDR3 of SEQ ID NO: 15, the LCDR1 of SEQ ID NO: 16, the LCDR2 of SEQ ID NO: 17, and the LCDR3 of SEQ ID NO: 18; and the HCDR1 of SEQ ID NO: 19, the HCDR2 of SEQ ID NO: 20, the HCDR3 of SEQ ID NO: 21, the LCDR1 of SEQ ID NO: 22, the LCDR2 of SEQ ID NO: 23, and the LCDR3 of SEQ ID NO: 24.

Furthermore, the sequences of the V_{H} and the V_{L} of the anti-meEGFR antibody or the antigen-binding fragment thereof of the present disclosure are selected from the group consisting of: the V_{H} including SEQ ID NO: 1 and the V_{L} including SEQ ID NO: 2; the V_{H} including SEQ ID NO: 3 and the V_{L} including SEQ ID NO: 4; and the V_{H} including SEQ ID NO: 5 and the V_{L} including SEQ ID NO: 6.

The anti-meEGFR antibody or the antigen-binding fragment thereof of the present disclosure can be selected from the group consisting of a single-domain antibody, a humanized antibody, a multimeric antibody, a single-chain variable fragment, a Fab fragment, a Fab′ fragment, and a F(ab′)2 fragment. In addition, the anti-meEGFR antibody of the present disclosure can be, for example, an IgG1, an IgG2, an IgG3, an IgG4, an IgM, an IgA1, an IgA2, an IgAsec, an IgD, or an IgE isotype antibody.

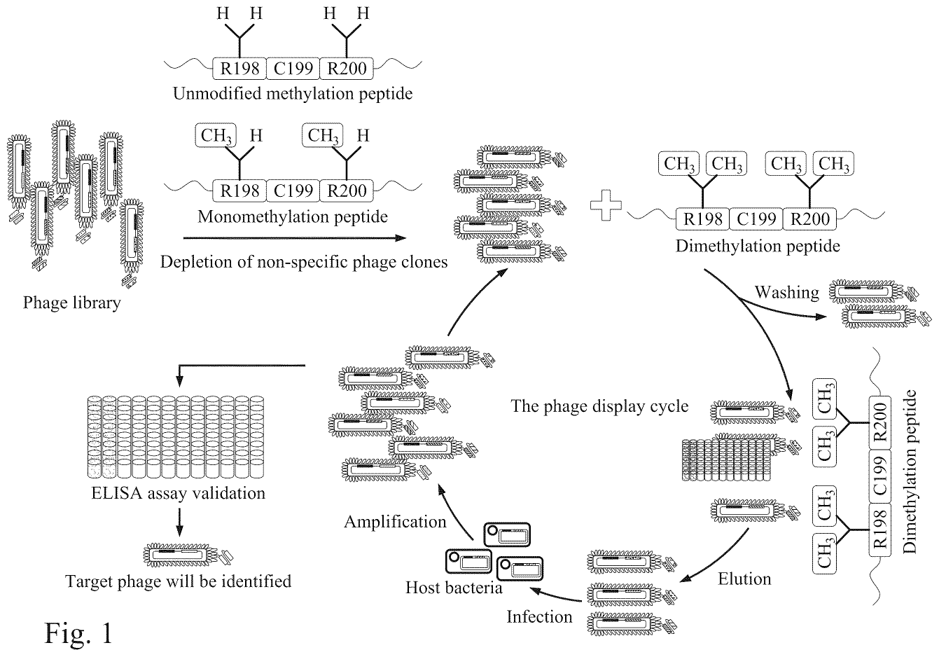

Reference is made to FIG. 1, which is a schematic diagram of the phage display technology for screening an anti-meEGFR antibody or an antigen-binding fragment thereof according to one aspect of the present disclosure. As shown in FIG. 1, the phage display technology derived from the human IgG library, and three anti-meEGFR antibodies or antigen-binding fragments thereof (hereinafter referred to as “Example 1”, “Example 2”, and “Example 3” respectively) with specificity to the meEGFR were isolated by stepwise screening with a R198/R200 asymmetrically dimethylated peptide (hereinafter referred to as “target peptide”). The sequences of the V_{H} and the V_{L} of Examples 1-3 are shown in Table 1, and the sequences of the HCDR1, the HCDR2, the HCDR3, the LCDR1, the LCDR2, and the LCDR3 of Examples 1-3 are shown in Table 2.

TABLE 1
The sequences of the V_{H} and the V_{L} in Examples 1-3

| Example | | Amino acid sequence | Nucleic acid sequence |
|---|---|---|---|
| Example 1 | V_{H} | SEQ ID NO: 1 | SEQ ID NO: 44 |
| | V_{L} | SEQ ID NO: 2 | SEQ ID NO: 45 |
| Example 2 | V_{H} | SEQ ID NO: 3 | SEQ ID NO: 46 |
| | V_{L} | SEQ ID NO: 4 | SEQ ID NO: 47 |
| Example 3 | V_{H} | SEQ ID NO: 5 | SEQ ID NO: 48 |
| | V_{L} | SEQ ID NO: 6 | SEQ ID NO: 49 |

TABLE 2
The sequences of the HCDR1, the HCDR2, the HCDR3,
the LCDR1, the LCDR2 and the LCDR3 in Examples 1-3

| Example | | Amino acid sequence | Nucleic acid sequence |
|---|---|---|---|
| Example 1 | HCDR1 | SEQ ID NO: 7 | SEQ ID NO: 50 |
| | HCDR2 | SEQ ID NO: 8 | SEQ ID NO: 51 |
| | HCDR3 | SEQ ID NO: 9 | SEQ ID NO: 52 |
| | LCDR1 | SEQ ID NO: 10 | SEQ ID NO: 53 |
| | LCDR2 | SEQ ID NO: 11 | SEQ ID NO: 54 |
| | LCDR3 | SEQ ID NO: 12 | SEQ ID NO: 55 |
| Example 2 | HCDR1 | SEQ ID NO: 13 | SEQ ID NO: 56 |
| | HCDR2 | SEQ ID NO: 14 | SEQ ID NO: 57 |
| | HCDR3 | SEQ ID NO: 15 | SEQ ID NO: 58 |
| | LCDR1 | SEQ ID NO: 16 | SEQ ID NO: 59 |
| | LCDR2 | SEQ ID NO: 17 | SEQ ID NO: 60 |
| | LCDR3 | SEQ ID NO: 18 | SEQ ID NO: 61 |
| Example 3 | HCDR1 | SEQ ID NO: 19 | SEQ ID NO: 62 |
| | HCDR2 | SEQ ID NO: 20 | SEQ ID NO: 63 |
| | HCDR3 | SEQ ID NO: 21 | SEQ ID NO: 64 |
| | LCDR1 | SEQ ID NO: 22 | SEQ ID NO: 65 |
| | LCDR2 | SEQ ID NO: 23 | SEQ ID NO: 66 |
| | LCDR3 | SEQ ID NO: 24 | SEQ ID NO: 67 |

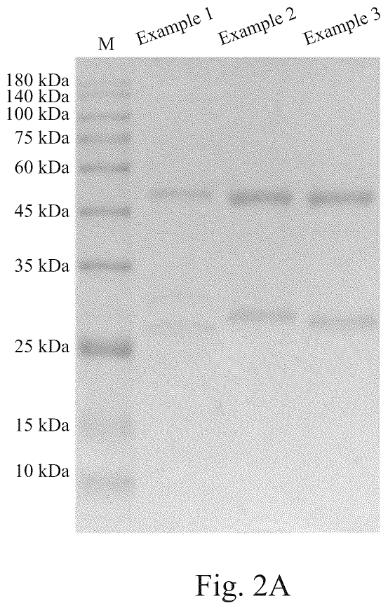

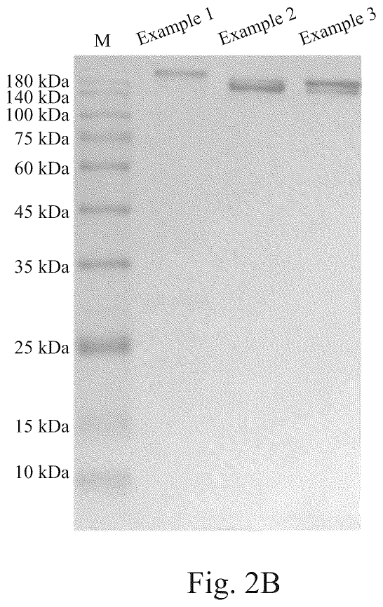

Reference is made to FIG. 2A and FIG. 2B, which show the sodium dodecyl sulfate polyacrylamide gel electrophoresis (SDS-PAGE) analysis results of Examples 1-3, where ‘M’ represents the protein molecular weight marker. The numbers on the left side of the figure indicate the corresponding molecular weights, and lanes 1-3 show the results of Examples 1-3, respectively. FIG. 2A shows the reduced SDS-PAGE results of the V_{H} and the V_{L} of Examples 1-3, while FIG. 2B shows the non-reduced SDS-PAGE results of Examples 1-3.

To further verify the affinity of Examples 1-3 for the meEGFR, ELISA analysis was used to test the binding affinity of Examples 1-3 with the target peptide and several different control peptides. The sequences and methylation modification positions of the target peptide and control peptides 1-5 are shown in Table 3. The target peptide has asymmetric dimethylation modifications at R198 and R200 residues. The control peptide 1 has no methylation modification, the control peptide 2 has monomethylation at R198 residue, the control peptide 3 has monomethylation at R200 residue, the control peptide 4 has symmetric dimethylation at R198 residue, and the control peptide 5 has symmetric dimethylation at R200 residue.

TABLE 3
The sequences of the target peptide and the control peptides

| Target | Sequence |
|---|---|
| Control peptide 1 | K(Biotin)QCSGRCRGKSPSDC-NH₂ |
| | SEQ ID NO: 25 |
| Target peptide | K(Biotin)QCSG[asymmetric dimethyl]RCR[asymmetric |
| | dimethyl]GKSPSDC-NH₂ |
| | SEQ ID NO: 26 |
| Control peptide 2 | K(Biotin)QCSG[monomethyl]RCRGKSPSDC-NH₂ |
| | SEQ ID NO: 27 |
| Control peptide 3 | K(Biotin)QCSGRCR[monomethyl]GKSPSDC-NH₂ |
| | SEQ ID NO: 28 |
| Control peptide 4 | K(Biotin)QCSG[symmetric dimethyl]RCRGKSPSDC-NH₂ |
| | SEQ ID NO: 29 |
| Control peptide 5 | K(Biotin)QCSGRCR[symmetric dimethyl]GKSPSDC-NH₂ |
| | SEQ ID NO: 30 |

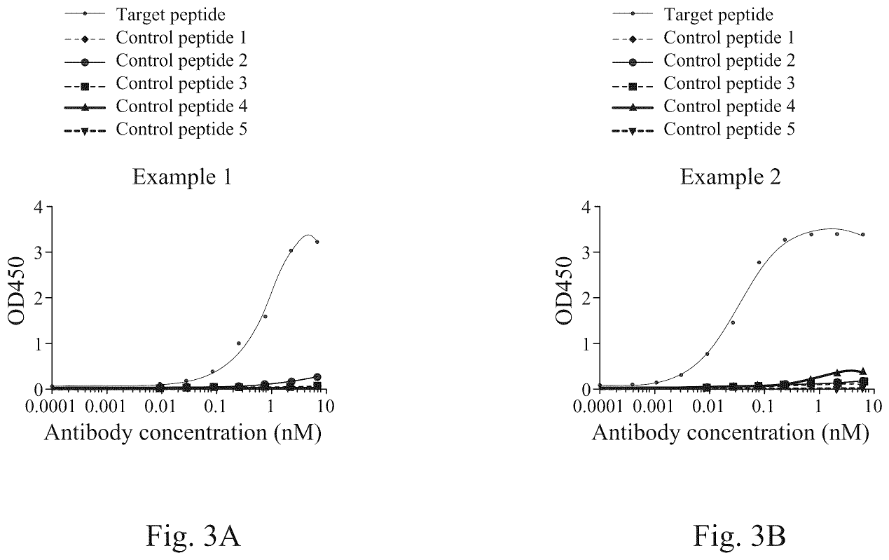

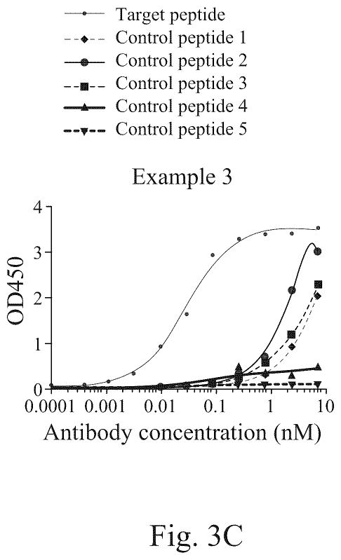

Reference is made to FIG. 3A to FIG. 3C and Table 4. FIG. 3A shows the binding affinity analysis results of Example 1 with the target peptide, FIG. 3B shows the binding affinity analysis results of Example 2 with the target peptide, FIG. 3C shows the binding affinity analysis results of Example 3 with the target peptide, and Table 4 shows the specificity and affinity analysis results of Examples 1-3 and the target peptide.

TABLE 4
The specificity and affinity analysis results
of Examples 1-3 and the target peptide

| | Specificity | | | Affinity | |
|---|---|---|---|---|---|
| | Target | Control | Control | EC₅₀ of target | |
| Example | peptide | peptide 4 | peptide 5 | peptide | |
| Example 1 | high | very low | very low | 1.712 | nM |
| Example 2 | high | low | low | 0.03589 | nM |
| Example 3 | high | medium | low | 0.02582 | nM |

The results show that Example 1, Example 2, and Example 3 all have high specificity for the target peptide, while they exhibit very low, low, or medium specificity for the control peptide 4 (symmetrically dimethylated at R198) and the control peptide 5 (symmetrically dimethylated at R200). The EC₅₀ of the target peptide in Example 1, Example 2, and Example 3 are 1.712 nM, 0.03589 nM, and 0.02582 nM, respectively, indicating high specificity and high affinity for the meEGFR. Example 1 was chosen for further testing due to its optimal specificity.

II. A Detection Kit and a Method for Detecting a meEGFR-Positive Cancer

The detection kit of the present disclosure is used to detect the meEGFR-positive cancer. The detection kit includes the anti-meEGFR antibody or the antigen-binding fragment thereof according to the foregoing aspect. The anti-meEGFR antibody or the antigen-binding fragment thereof can be conjugated with a label. The label refers to a substance that can be covalently bound or be physically adsorbed to the anti-meEGFR antibody or the antigen-binding fragment thereof of the present disclosure, and can thus be used to detect the presence of the anti-meEGFR antibody or the antigen-binding fragment thereof. The label can be a fluorescent label, a chemiluminescent label, a radioactive isotope label, an enzyme label, a biotin label, or a combination thereof. Furthermore, the fluorescent label includes, but is not limited to, a fluorescent group such as FAM, JOE, or VIC. The chemiluminescent label includes, but is not limited to, an electrochemiluminescent compound or a chemiluminescent compound, such as luminol, isoluminol, or acridinium salts. The radioactive isotope label includes, but is not limited to, ³H, ¹⁴C, ³²P, ³⁵S, ¹²⁵⁽¹³¹⁾I, ⁷⁵Se. The enzyme label includes, but is not limited to, an enzyme with a detectable product, such as luciferase, peroxidase, alkaline phosphatase, β-galactosidase, and similar substance.

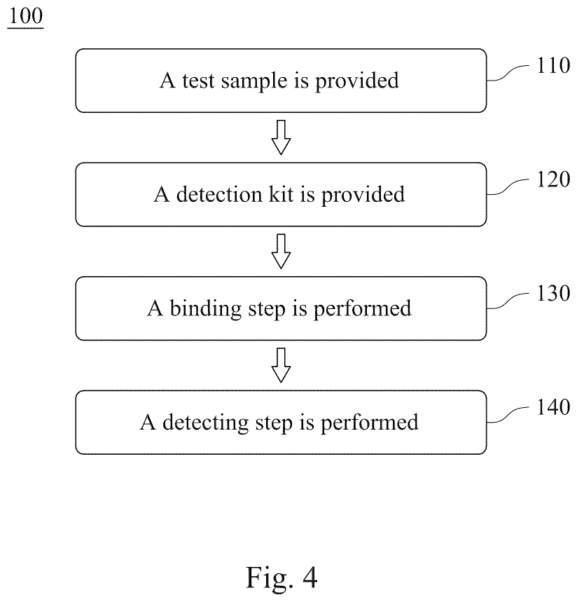

The detection kit of the present disclosure includes the anti-meEGFR antibody or the antigen-binding fragment thereof, which can be used to detect the meEGFR-positive cancer. The meEGFR-positive cancer can be a breast cancer, a colorectal cancer, a prostate cancer, a lung cancer, or a pancreatic cancer. Reference is made to FIG. 4, which is a step flow chart of the method for detecting the meEGFR-positive cancer 100 according to another aspect of the present disclosure. The method for detecting the meEGFR-positive cancer 100 includes Step 110, Step 120, Step 130, and Step 140.

In Step 110, a test sample obtained from a subject is provided. The test sample can be a frozen tissue section, a paraffin-embedded tissue section, or a tissue microarray.

In Step 120, the detection kit of the present disclosure is provided. The detection kit includes the anti-meEGFR antibody or the antigen-binding fragment thereof, and the anti-meEGFR antibody or the antigen-binding fragment thereof can conjugate with the label. The label can be the fluorescent label, the chemiluminescent label, the radioactive isotope label, the enzyme label, the biotin label, or a combination thereof.

In Step 130, a binding step is performed, wherein the test sample is contacted with the anti-meEGFR antibody or the antigen-binding fragment thereof to form a binding reaction.

In Step 140, a detection step is performed to detect whether the test sample includes an antibody-cancer cell complex. When there is the antibody-cancer cell complex in the test sample, it can be determined that the subject providing the test sample is a meEGFR-positive cancer patient. The term “antibody-cancer cell complex” refers to a complex formed by the specific binding of the anti-meEGFR antibody or the antigen-binding fragment thereof of the present disclosure to cancer cells expressing the meEGFR. The method for detecting the presence of the antibody-cancer cell complex includes, but is not limited to, immunofluorescence, immunohistochemistry, Western blotting, enzyme-linked immunosorbent assay (ELISA), or autoradiography.

Based on the method for detecting the meEGFR-positive cancer 100 of the present disclosure, if a subject is determined not to be a meEGFR-positive cancer patient, the subject can still be considered for subsequent treatment with a clinically approved EGFR-TKI. However, if the subject is determined to be the meEGFR-positive cancer patient, other treatment methods need to be used, such as using the anti-meEGFR antibody or the antigen-binding fragment thereof of the present disclosure, a pharmaceutical composition for a treating cancer of the present disclosure, a meEGFR-specific chimeric antigen receptor expressing cell of the present disclosure, an innate cell engager of the present disclosure, and/or an antibody conjugate of the present disclosure.

In the experiment, the detection kit of the present disclosure is further used to detect whether the test sample includes the antibody-cancer cell complex, as a basis for whether the subject is the meEGFR-positive cancer patient. In the following experimental examples, the anti-meEGFR antibody or the antigen-binding fragment thereof in the detection kit is Example 1, and the presence of the antibody-cancer cell complex in the test sample was detected using immunohistochemistry and immunofluorescence staining.

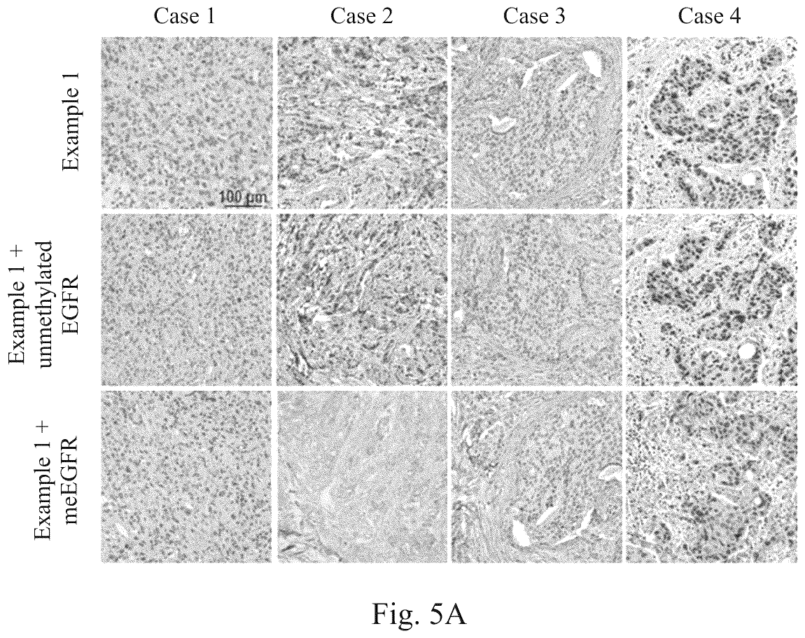

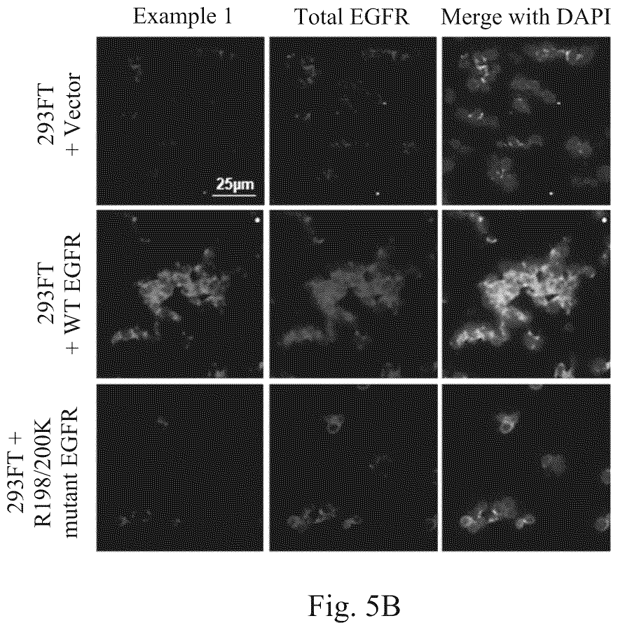

Reference is made to FIG. 5A and FIG. 5B, which show the binding specificity analysis results of the detection kit according to still another aspect of the present disclosure. FIG. 5A shows the immunohistochemistry analysis results of human breast cancer tissue samples using the detection kit of the present disclosure in the absence or presence of the meEGFR (hot peptide) or unmethylated EGFR (cold peptide). FIG. 5B shows the immunofluorescence staining analysis results of 293 FT cells transfected with a vector, a plasmid encoding wild-type EGFR or a plasmid encoding R198/200K mutant EGFR (unmethylated mutant) using the detection kit of the present disclosure. The results of FIG. 5A show that the detection kit of the present disclosure can detect the antibody-cancer cell complex in the presence of the meEGFR, while the results of FIG. 5B show that the antibody-cancer cell complex cannot be detected when EGFR is the unmethylated mutant. These results indicate that the detection kit of the present disclosure has high specificity and high affinity for the meEGFR.

### III. A Pharmaceutical Composition for Treating a Cancer

The pharmaceutical composition for treating the cancer of the present disclosure includes the anti-meEGFR antibody or the antigen-binding fragment thereof of the present disclosure and a pharmaceutically acceptable carrier. Additionally, the pharmaceutical composition for treating the cancer can further include a chemotherapeutic agent, an immunomodulator, a targeted therapeutic drug, an antibody drug, or a combination thereof, wherein the antibody drug is different from the anti-meEGFR antibody or the antigen-binding fragment thereof.

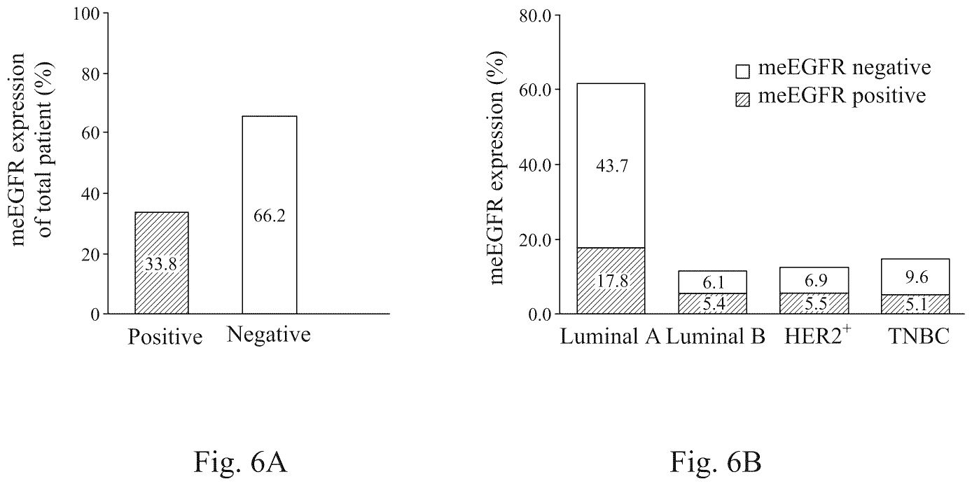

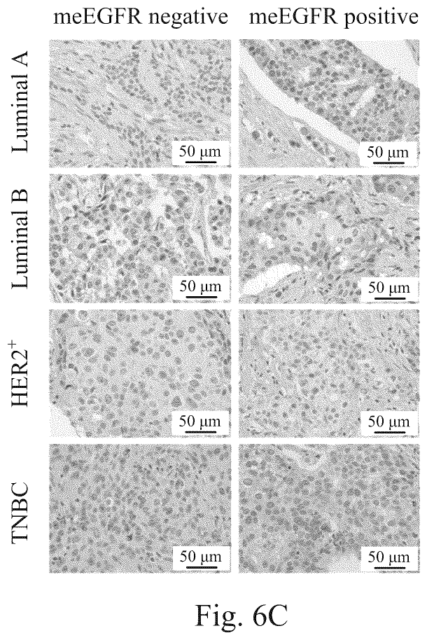

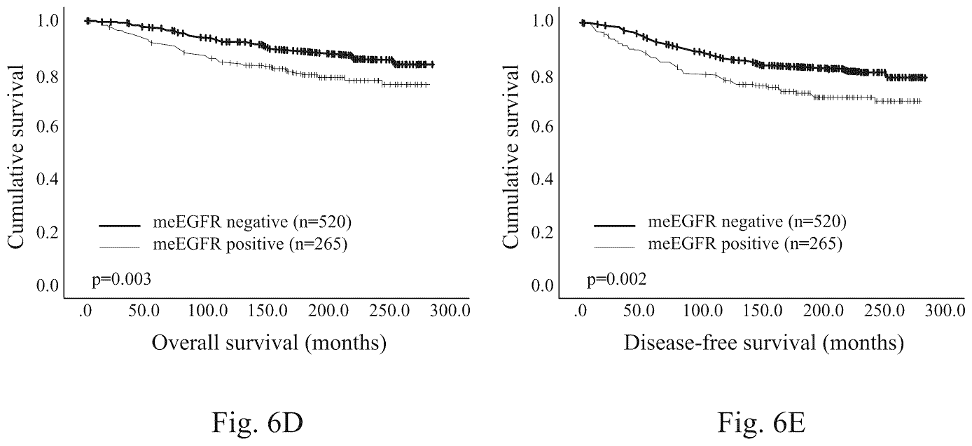

A total of 785 breast cancer patients were recruited for testing, and the tumor tissues provided by the breast cancer patients were tested using the detection kit of the present disclosure and the method for detecting the meEGFR-positive cancer of the present disclosure. Reference is made to FIG. 6A to FIG. 6C, which show the analysis results of the meEGFR expression in the breast cancer patients. The results of FIG. 6A show that approximately 33.8% of the tumor tissues of the breast cancer patients are positive for the meEGFR expression. Further analysis of the meEGFR expression in different types of the breast cancer patients, as shown in FIG. 6B and FIG. 6C, indicates that the ratio of the meEGFR-positive to the meEGFR-negative in luminal A breast cancer patients is 17.8%: 43.7%, while the ratios in luminal B breast cancer patients, human epidermal growth factor receptor 2 (HER2)-positive (HER2+) breast cancer patients, and triple negative breast cancer (TNBC) patients are 5.4%: 6.1%, 5.5%: 6.9%, and 5.1%: 9.6%, respectively. These results support that the meEGFR is an important oncogenic marker in the breast cancer patients.

The experiment further analyzed the correlation between the meEGFR expression and clinical prognosis, using two time-to-event endpoints: overall survival (OS) and disease free survival (DFS). The evaluation indicator of overall survival is to define “death” as an event and observe the time from the start of the clinical trial to death, while evaluation indicator of disease-free survival is to define “tumor recurrence” as an event and observes the time from the start of the clinical trial to tumor recurrence.

Reference is made to FIG. 6D and FIG. 6E. FIG. 6D shows the analysis results of overall survival in the meEGFR-positive patient and the meEGFR-negative breast cancer patients, and FIG. 6E shows the analysis results of disease-free survival in the meEGFR-positive patients and the meEGFR-negative breast cancer patients. The number of the meEGFR-positive breast cancer patients analyzed is 265, and the number of the meEGFR-negative breast cancer patients analyzed is 520. The results of FIG. 6D and FIG. 6E show that meEGFR-negative breast cancer patients have higher overall survival and disease-free survival time compared to the meEGFR-positive breast cancer patients. The p-value for overall survival is 0.003, and the p-value for disease-free survival is 0.002, as evaluated by the log-rank test. These results further confirm that meEGFR is an important oncogenic marker in the breast cancer patients.

The experiment further evaluated the therapeutic effect of the pharmaceutical composition for treating the cancer of the present disclosure on the meEGFR-positive cancer. In the following experimental examples, the anti-meEGFR antibody or the antigen-binding fragment thereof in the pharmaceutical composition for treating the cancer is Example 1. Different tumor cells were treated with Example 1 or cetuximab, and the cell viability of the tumor cells was analyzed. The tumor cells used were different human breast cancer cell lines, including SUM159PT, BT549, MCF7, MDA-MB-468, HCC1806, and HCC1954. SUM159PT, BT549, MDA-MB-468, and HCC1806 are triple negative breast cancer cells with negative expression of estrogen receptor (ER), progesterone receptor (PR), and HER2. MCF7 is a tubular breast cancer cell line with positive ER expression but low EGFR expression. HCC1954 is a breast cancer cell line with overexpression of HER2 but negative expression of ER and PR. The tumor cell lines used were purchased from the American Type Culture Collection (ATCC).

In the experiment, SUM159PT and BT549 were seeded at a density of 5×10³ cells/well, HCC1954, HCC1806, and MDA-MB-468 were seeded at a density of 8×10³ cells/well, and MCF7 was seeded at a density of 1×10⁴ cells/well in 96-well plates. After 16-18 hours of culture, the culture medium in the wells was removed, and DMEM-F12 medium containing different concentrations of Example 1 or cetuximab (concentrations of 1 nM, 10 nM, 100 nM, and 1000 nM respectively) was added. After 48 hours of culture, an MTT assay was performed. The steps of the MTT assay were removing the culture medium from the 96-well plates, adding 100 μL of DMEM-F12 medium containing 5 μg/mL MTT, incubating in the incubator for 4 hours, completely removing the solution in the wells, adding 100 μL of DMSO to dissolve the crystals, and reading the absorbance at 570 nm using an enzyme immunoassay analyzer.

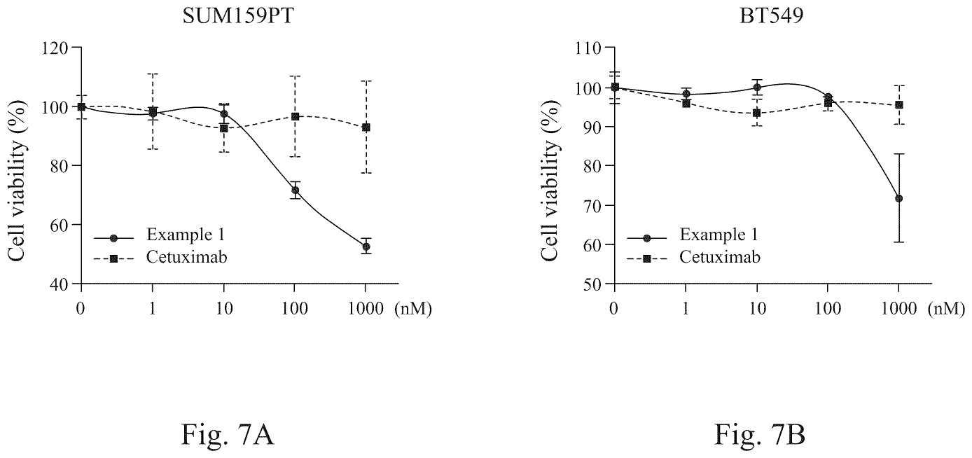

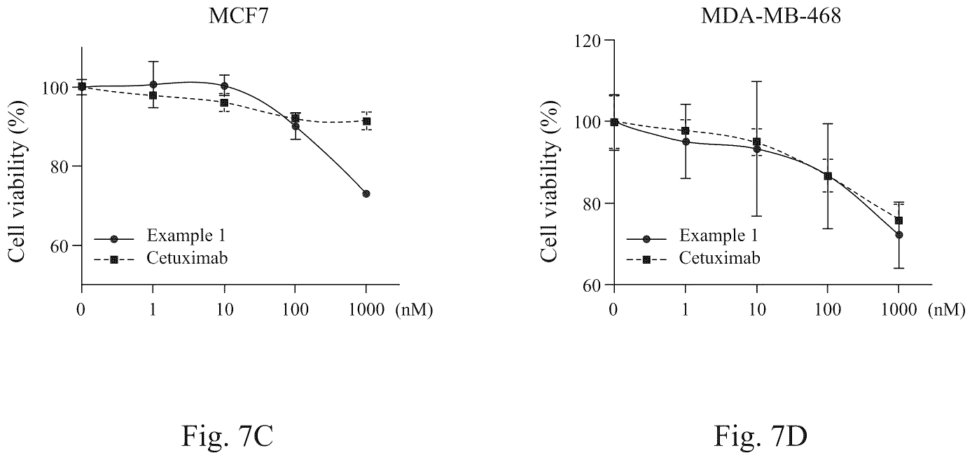

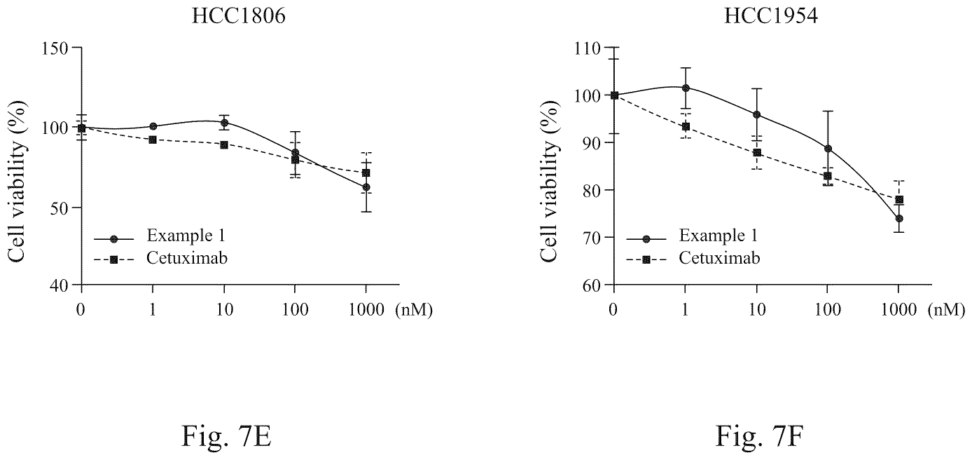

Reference is made to FIG. 7A to FIG. 7F, which show the analysis results of tumor cell death induced by the pharmaceutical composition for treating the cancer according to one embodiment of yet another aspect of the present disclosure. FIG. 7A shows the analysis results of SUM159PT, FIG. 7B shows the analysis results of BT549, FIG. 7C shows the analysis results of MCF7, FIG. 7D shows the analysis results of MDA-MB-468, FIG. 7E shows the analysis results of HCC1806, and FIG. 7F shows the analysis results of HCC1954. The results show that when the concentration of Example 1 is 1000 nM, the pharmaceutical composition for treating the cancer of the present disclosure achieves better inhibitory effect on growth of the tumor cell than cetuximab in SUM159PT, BT549, and MCF7, and achieves comparable inhibitory effect on growth of the tumor cell to cetuximab in MDA-MB-468, HCC1806, and HCC1954.

Further, Example 1 was combined with the chemotherapy drug as another embodiment of the pharmaceutical composition for treating the cancer of the present disclosure, and the inhibitory effect on growth of tumor cell was analyzed. The tumor cell used was SUM159PT, and the chemotherapeutic drugs used were 5-fluorouracil and gemcitabine. In the experiment, separate treatments with the chemotherapy drug alone or cetuximab combined with the chemotherapy drug were used as comparative examples. The treatment strategies of Examples 4-7 and Comparative Examples 1-6 are shown in Table 5. The control was SUM159PT that was not treated with any antibodies or chemotherapeutic drugs. After SUM159PT was treated with Comparative 10 Examples 1-6 and Examples 4-7 respectively, the cell viability assay was performed.

TABLE 5
The treatment strategies of Examples
4-7 and Comparative Examples 1-6

| | | Concentration | | Chemotherapeutic |
|---|---|---|---|---|
| Group | Antibody | of antibody | | drug |
| Comparative | — | — | | 5-fluorouracil |
| Example 1 | | | | |
| Comparative | Cetuximab | 0.1 | μM | 5-fluorouracil |
| Example 2 | | | | |
| Comparative | Cetuximab | 1 | μM | 5-fluorouracil |
| Example 3 | | | | |
| Comparative | — | — | | Gemcitabine |
| Example 4 | | | | |
| Comparative | Cetuximab | 0.1 | μM | Gemcitabine |
| Example 5 | | | | |
| Comparative | Cetuximab | 1 | μM | Gemcitabine |
| Example 6 | | | | |
| Example 4 | Example 1 | 0.1 | μM | 5-fluorouracil |
| Example 5 | Example 1 | 1 | μM | 5-fluorouracil |
| Example 6 | Example 1 | 0.1 | μM | Gemcitabine |
| Example 7 | Example 1 | 1 | μM | Gemcitabine |

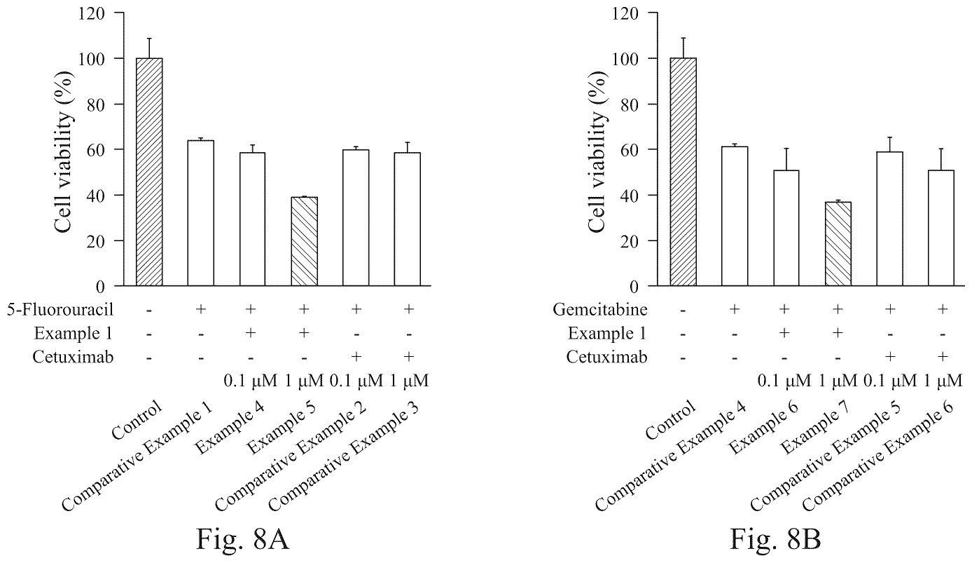

Reference is made to FIG. 8A and FIG. 8B, which show the analysis results of SUM159PT cell death induced by the pharmaceutical composition for treating the cancer according to yet another embodiment of another aspect of the present disclosure. The results in FIG. 8A show that in Comparative Example 2 and Comparative Example 3 with the combined treatment of cetuximab and 5-fluorouracil, no corresponding inhibitory effect was observed when the concentration of cetuximab was increased. In contrast, in Example 4 and Example 5 with the combined treatment of Example 1 and 5-fluorouracil, the inhibitory effect on growth of SUM159PT was concentration-dependent. Among all groups, Example 5 had the best inhibitory effect on growth of SUM159PT. The results in FIG. 8B show that in Comparative Example 5 and Comparative Example 6 with the combined treatment of cetuximab and gemcitabine, the inhibitory effect on growth of SUM159PT was concentration-dependent when the concentration of cetuximab was increased. Similarly, in Example 6 and Example 7 with the combined treatment of Example 1 and gemcitabine, the inhibitory effect on growth of SUM159PT was concentration-dependent. Among all groups, Example 7 had the best inhibitory effect on growth of SUM159PT. The results indicate that the simultaneous administration of the anti-meEGFR antibody or the antigen-binding fragment thereof of the present disclosure and the chemotherapeutic drug has a synergistic effect, significantly inhibiting tumor cell growth.

IV. A meEGFR-specific chimeric antigen receptor, a nucleic acid, a meEGFR-specific chimeric antigen receptor expressing cell, and a pharmaceutical composition for treating a cancer.

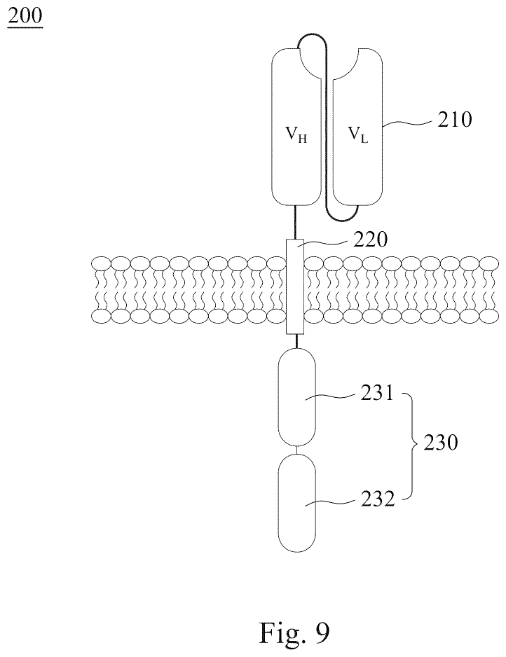

Reference is made to FIG. 9, which is a theoretical structural diagram of a meEGFR-specific chimeric antigen receptor 200 according to still another aspect of the present disclosure. The meEGFR-specific chimeric antigen receptor 200 of the present disclosure includes an extracellular domain 210 and an intracellular signaling domain 230.

The extracellular domain 210 is for recognizing the meEGFR, and the meEGFR has asymmetric dimethylation modifications at R198 and R200 residues. The extracellular domain 210 includes the anti-meEGFR antibody or the antigen-binding fragment thereof of the present disclosure.

Furthermore, the meEGFR-specific chimeric antigen receptor 200 can further include a transmembrane domain 220, the transmembrane domain 220 links the extracellular domain 210 and the intracellular signaling domain 230. The transmembrane domain 220 can include a transmembrane portion of a co-stimulatory molecule, such as the transmembrane portion of a T cell co-stimulatory molecule. Specifically, the transmembrane domain 220 can be CD137 (4-1BB), T cell receptor α chain, T cell receptor β chain, CD2, CD30, CD38, CD3γ, CD4, CD7, CD8a, CD8B, CD11a (ITGAL), CD11b (ITGAM), CD11c (ITGAX), CD11d (ITGAD), CD18 (ITGB2), CD19 (B4), CD27 (TNFRSF7), CD28, CD29 (ITGB1), CD30 (TNFRSF8), CD40 (TNFRSF5), CD48 (SLAMF2), CD49a (ITGA1), CD49d (ITGA4), CD49f (ITGA6), CD66a (CEACAM1), CD66b (CEACAM8), CD66c (CEACAM6), CD66d (CEACAM3), CD66e (CEACAM5), CD69 (CLEC2), CD79A, CD79B, CD84 (SLAMF5), CD96 (Tactile), CD100 (SEMA4D), CD103 (ITGAE), CD134 (OX40), CD150 (SLAMF1), CD158A (KIR2DL1), CD158B1 (KIR2DL2), CD158B2 (KIR2DL3), CD158C (KIR3DP1), CD158D (KIRDL4), CD158F1 (KIR2DL5A), CD158F2 (KIR2DL5B), CD158K (KIR3DL2), CD160 (BY55), CD162 (SELPLG), CD226 (DNAM1), CD229 (SLAMF3), CD244 (SLAMF4), CD247 (CD3ζ), CD258 (LIGHT), CD268 (BAFFR), CD270 (TNFSF14), CD272 (BTLA), CD276 (B7-H3), CD279 (PD-1), CD314 (NKG2D), CD319 (SLAMF7), CD335 (NK-p46), CD336 (NK-p44), CD337 (NK-p30), CD352 (SLAMF6), CD353 (SLAMF8), CD355 (CRTAM), CD357 (TNFRSF18), inducible T cell co-stimulatory molecule (ICOS), LFA-1 (CD11a/CD18), NKG2C, DAP-10, ICAM-1, NKp80 (KLRF1), IL-2Rβ, IL-2Rγ, IL-7Rα, LFA-1, SLAMF9, LAT, GADS (GrpL), SLP-76 (LCP2), PAG1/CBP, CD83 ligand, Fcγ receptor, MHC class I molecule, MHC class II molecule, TNF receptor protein, immunoglobulin, interleukin receptor, integrin, activated NK cell receptor, Toll-like receptor, or a combination thereof.

The intracellular signaling domain 230 can include a co-stimulatory domain 231 and a primary signaling domain 232. The intracellular signaling domain 230 can be CD137 (4-1BB), activated NK cell receptor, CD276 (B7-H3), BAFFR, BLAME (SLAMF8), BTLA, CD100 (SEMA4D), CD103, CD160 (BY55), CD18, CD19, CD19a, CD2, CD247, CD27, CD28, CD29, CD3δ, CD3ε, CD3γ, CD30, CD4, CD40, CD49a, CD49D, CD49f, CD69, CD7, CD84, CD8α, CD8β, CD96 (Tactile), CD11a, CD11b, CD11c, CD11d, CDS, CEACAM1, CRTAM, interleukin receptor, DAP-10, DNAM1 (CD226), Fcγ receptor, GADS, GITR, HVEM (LIGHTR), IA4, ICAM-1, Igα (CD79a), IL2Rβ, IL2Rγ, IL7Rα, immunoglobulin-like protein, inducible T cell co-stimulatory molecule (ICOS), integrin, ITGA4, ITGA6, ITGAD, ITGAE, ITGAL, ITGAM, ITGAX, ITGB2, ITGB7, ITGB1, KIRDS2, LAT, LFA-1, specific binding CD83 ligand, LIGHT (TNFSF14), LTBR, Ly9 (CD229), lymphocyte function-associated antigen-1 (LFA-1 (CD11a/CD18)), MHC class I molecule, NKG2C, NKG2D, NKp30, NKp44, NKp46, NKp80 (KLRF1), OX-40, PAG/Cbp, CD279 (PD-1), PSGL1, SELPLG (CD162), signaling lymphocyte activation molecule (SLAM protein), SLAMF1 (CD150; IPO-3), SLAMF4 (CD244; 2B4), SLAMF6 (NTB-A; Ly108), SLAMF7, SLP-76, TNF receptor protein, TNFR2, Toll-like receptor, TRANCE/RANKL, VLA1, VLA-6, or a combination thereof.

The nucleic acid of the present disclosure encodes the meEGFR-specific chimeric antigen receptor according to the foregoing aspect. The term “nucleic acid” can include naturally occurring and/or non-naturally occurring nucleotides and bases, such as those with backbone modifications, and can include naturally occurring and/or non-naturally occurring nucleotides, including but not limited to DNA, RNA, and PNA. The term “nucleic acid sequence” refers to the linear sequence of nucleotides that compose a nucleic acid molecule or polynucleotide.

The nucleic acid of the present disclosure includes, in sequence from the 5′ end to the 3′ end, an extracellular domain coding fragment and an intracellular signaling domain coding fragment. The extracellular domain coding fragment includes the nucleic acid encoding the anti-meEGFR antibody or the antigen-binding fragment thereof of the present disclosure, which includes a V_{H} coding fragment and a V_{L} coding fragment. The V_{H} coding fragment includes the nucleic acid sequences shown in SEQ ID NO: 44, SEQ ID NO: 46, or SEQ ID NO: 48, and the V_{L} coding fragment includes the nucleic acid sequences shown in SEQ ID NO: 45, SEQ ID NO: 47, or SEQ ID NO: 49. In addition, the nucleic acid encoding the anti-meEGFR antibody or the antigen-binding fragment thereof of the present disclosure includes one or more CDR coding fragments selected from SEQ ID NO: 50 to SEQ ID NO: 67.

Furthermore, the nucleic acid can further include a transmembrane domain coding fragment, which links the extracellular domain coding fragment and the intracellular signaling domain coding fragment. The intracellular signaling domain coding fragment can include a co-stimulatory domain coding fragment and a primary signaling domain coding fragment.

The meEGFR-specific chimeric antigen receptor expressing cell of the present disclosure includes an immune cell and the nucleic acid of the present disclosure. The meEGFR-specific chimeric antigen receptor expressing cell is obtained by transfecting the nucleic acid of the present disclosure into the immune cell. The immune cell can be a T cell or a natural killer cell. The meEGFR-specific chimeric antigen receptor expressing cell of the present disclosure can specifically recognize the meEGFR on the tumor cell membrane and exert cytotoxic effect on tumor cells. Therefore, the meEGFR-specific chimeric antigen receptor expressing cell of the present disclosure can be used in combination with a pharmaceutically acceptable carrier as another aspect of the pharmaceutical composition for treating the cancer.

In the experiment, the meEGFR-specific chimeric antigen receptor of Example 8 (hereinafter referred to as “Example 8”) was prepared as an example, which includes an IgG K leader sequence (the sequence referenced as SEQ ID NO: 31), a scFv of the anti-meEGFR antibody of the present disclosure (the sequence referenced as SEQ ID NO: 32), a CD28 hinge region (the sequence referenced as SEQ ID NO: 33), a CD28 transmembrane domain (the sequence referenced as SEQ ID NO: 34), a CD28 co-stimulatory domain (the sequence referenced as SEQ ID NO: 35), and a CD33 primary signaling domain (the sequence referenced as SEQ ID NO: 36). The nucleic acid encoding Example 8 was constructed using a lentiviral vector and subsequently packaged into corresponding lentiviral particles. The lentiviral particles were used to transduce the nucleic acid encoding Example 8 into NK-92 cell line (hereinafter referred to as “NK-92”) to obtain the meEGFR-specific chimeric antigen receptor expressing cell of Example 9 (hereinafter referred to as “Example 9”), and the cell cytotoxicity assay of Example 9 was conducted.

The nucleic acid encoding Example 8 includes a IgG K leader sequence coding fragment (the sequence referenced as SEQ ID NO: 37), a scFv coding fragment of the anti-meEGFR antibody (the sequence referenced as SEQ ID NO: 38), a CD28 hinge region coding fragment (the sequence referenced as SEQ ID NO: 39), a CD28 transmembrane domain coding fragment (the sequence referenced as SEQ ID NO: 40), a CD28 co-stimulatory domain coding fragment (the sequence referenced as SEQ ID NO: 41), and a CD34 primary signaling domain coding fragment (the sequence referenced as SEQ ID NO: 42).

The experiment was divided into two groups. The tumor cells used in one group were the human colorectal cancer cell line SW620 (hereinafter referred to as “SW620”) transfected with a plasmid encoding wild-type PRMT1, and the tumor cells used in the other group were SW620 transfected with a plasmid encoding a mutant PRMT1 (hereinafter referred to as “mutant PRMT1”) that loses enzymatic activity. The tumor cells were co-cultured with Example 9 at a ratio of 2:1 in a humidified incubator with the IncuCyte® Zoom system (Essen Bioscience) for 24 hours. The cell cytotoxicity assay was measured using the IncuCyte® Zoom system according to the manufacturer's instruction, and apoptosis was assessed by measuring the area with fluorescent signals using the IncuCyte® Caspase-3/7 green apoptosis detection reagent, where green fluorescent signals represent apoptotic cell populations.

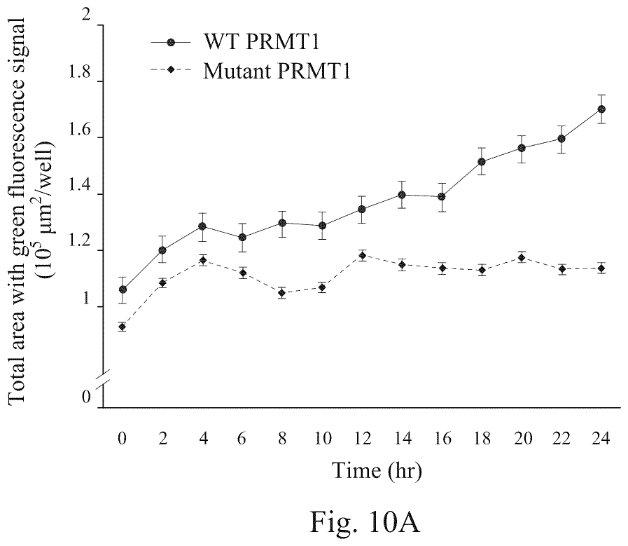

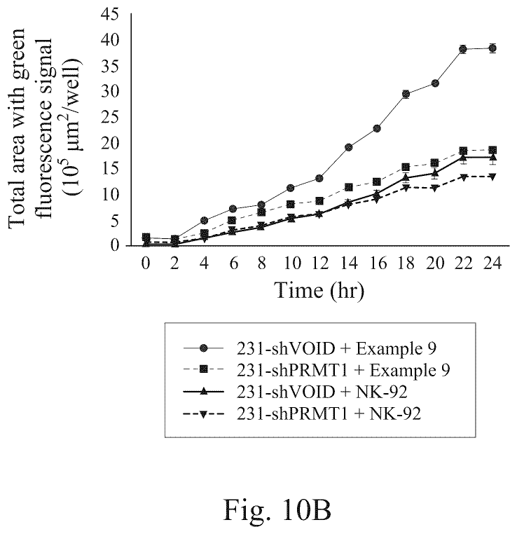

Reference is made to FIG. 10A, which shows the analysis results of the specific lysis of SW620 by Example 9. The results indicate that the introduction of wild-type PRMT1 into SW620 increases the methylation modification of EGFR, thereby enhancing the cytotoxic effect of Example 9 on SW620, while the group introduced mutant PRMT1 is a negative control.

The ability of Example 9 to induce MDA-MB-231 cell death was also tested. In the experiment, MDA-MB-231 was first treated with a shRNA targeting PRMT1 (shPRMT1) and a control shRNA (shVOID), respectively. The MDA-MB-231 treated with shPRMT1 (hereinafter referred to as “231-shPRMT1”) had reduced methylation modification of EGFR due to the knockout of PRMT1, while the methylation modification of EGFR in the MDA-MB-231 treated with shVOID (hereinafter referred to as “231-shVOID”) was not affected. The day before the experiment, 231-shPRMT1 and 231-shVOID were respectively seeded into 96-well plates at a density of 5×10³ cells per well. After 16-18 hours, the culture medium was removed, and 2.5×10³ Example 9 or NK-92 cells were added to each well for co-culture with the tumor cells. In each well, DMEM-F12 medium containing the IncuCyte® Caspase-3/7 green apoptosis detection reagent (250× dilution) was added. The plates were placed in the IncuCyte® Zoom system, with images taken every 2 hours under both brightfield and fluorescence conditions for a total of 24 hours. The analysis was then performed using software of the IncuCyte® Zoom system to determine whether the presence of EGFR methylation modification affects the cytotoxicity effect of Example 9 on MDA-MB-231.

Reference is made to FIG. 10B, which shows the analysis results of the specific lysis of MDA-MB-231 by Example 9. The results indicate that Example 9 has the best cytotoxic effect on 231-shVOID with normal EGFR methylation modification, while the cytotoxic effect of Example 9 on 231-shPRMT1 with reduced EGFR methylation modification is similar to that of the native NK-92.

These results indicate that the meEGFR-specific chimeric antigen receptor expressed by the meEGFR-specific chimeric antigen receptor expressing cell of the present disclosure can specifically bind to the meEGFR on the tumor cells, endowing the meEGFR-specific chimeric antigen receptor expressing cell with an immunotoxic activity, thereby effectively killing tumor cells. Particularly, when the tumor cells exhibit normal levels of EGFR methylation modification, the tumor cells are sensitive to the cytotoxic effect of the meEGFR-specific chimeric antigen receptor expressing cell of the present disclosure.

### V. Innate Cell Engager

The innate cell engager of the present disclosure includes a meEGFR antigen recognition domain and at least one immune cell receptor binding domain. The meEGFR antigen recognition domain includes the anti-meEGFR antibody or the antigen-binding fragment thereof of the present disclosure, and the at least one immune cell receptor binding domain has binding specificity for one or more of CD3, CD8, CD16, CD19, or NKG2D.

The at least one immune cell receptor binding domain can specifically bind to a receptor on various types of the immune cell. The immune cell can be selected from the group consisting of a T cell, a natural killer cell, a B cell, a dendritic cell, a monocyte, a macrophage, a neutrophil, a mesenchymal stem cell, and a neural stem cell.

Furthermore, the innate cell engager of the present disclosure can be a bispecific T cell engage, a trispecific T cell engager, or a multispecific T cell engager. The innate cell engager can also be a bispecific natural killer cell engager, a trispecific natural killer cell engager, or a multispecific natural killer cell engager.

The innate cell engager of the present disclosure can be based on the structure of scFv, and connect V_{L} and/or V_{H} with a short linker peptide. The linker peptide forms a bridge of about 3.5 nm between the carboxyl terminus of one variable domain and the amino terminus of another variable domain. For example, the meEGFR antigen recognition domain of the innate cell engager of the present disclosure can be an scFv structure including V_{L}-linker peptide-V_{H} or V_{H}-linker peptide-V_{L}. The sequence of the V_{H} can be selected from the group consisting of SEQ ID NO: 1, SEQ ID NO: 3, and SEQ ID NO: 5, and the sequence of the V_{L} can be selected from the group consisting of SEQ ID NO: 2, SEQ ID NO: 4, and SEQ ID NO: 6. The linker peptide can be a repeat fragment of the sequence referenced as SEQ ID NO: 43, which is (GGGGS)n or variants thereof, where n is a non-zero natural number, preferably in the range of 1 to 20.

The innate cell engager of the present disclosure can be obtained through the following method: constructing an expression vector using the nucleic acid encoding the anti-EGFR antibody or the antigen-binding fragment thereof of the present disclosure and the nucleic acid encoding the anti-CD3 antibody, introducing the constructed expression vector into CHO cells or HEK293 cells to obtain transfected cells. Then, the obtained transfected cells are cultured, and the culture medium is purified by chromatography to obtain the innate cell engager of the present disclosure.

### VI. Antibody Conjugate

The antibody conjugate of the present disclosure include the anti-meEGFR antibody or the antigen-binding fragment thereof and an effector molecule. The effector molecule is conjugated with the anti-meEGFR antibody or the antigen-binding fragment thereof through a chemical bond or a linker, wherein the effector molecule can be a toxin, a growth inhibitor, toxic protein, a radionuclide, a radioactive isotope, a chemotherapeutic drug, or a combination thereof.

Furthermore, the conjugation between the anti-meEGFR antibody or the antigen-binding fragment thereof and the effector molecule of the present disclosure can be achieved by a conjugating agent, wherein the conjugating agent can be one or more of a non-selective conjugating agent, a carboxyl conjugating agent, a peptide chain, and a disulfide bond conjugating agent. The non-selective conjugating agent refers to a compound that forms a covalent bond between the effector molecule and the anti-meEGFR antibody or the antigen-binding fragment thereof of the present disclosure, such as glutaraldehyde. The carboxyl conjugating agent can be one or more of a maleimide anhydride conjugating agent (such as maleimide anhydride) and an acyl hydrazone conjugating agent (conjugation site being acyl hydrazone).

The linker can be a degradable linker or a non-degradable linker, wherein the degradable linker is usually easily degraded in the intracellular environment, such as at the target site, thereby releasing the drug. A suitable degradable linker includes an enzyme-degradable linker, which includes a peptide linker that can be degraded by intracellular protease (such as lysosomal protease or endosomal protease) or a glycosidic linker. The peptide linker can be a dipeptide, such as valine-citrulline, phenylalanine-lysine, or valine-alanine. The glycosidic linker can be a linker including glucoside that can be degraded by glucosidase. Other suitable degradable linkers include a pH-sensitive linker (such as hydrazone linkers that hydrolyze at pH less than 5.5) and a linker that degrade under reducing condition (such as a disulfide bond linker). The non-degradable linker usually releases the drug under condition where the antibody is hydrolyzed by the protease.

The toxin can be a small molecule toxin or an enzyme-active toxin, such as but not limited to diphtheria toxin λ chain, non-binding active fragment of diphtheria toxin, exotoxin λ chain, ricin λ chain, abrin λ chain, modeccin λ chain, alpha-sarcin, Aleurites fordii protein, dianthin protein, Phytolaca americana protein, Momordica charantia inhibitor, curcin, crotin, Saponaria officinalis inhibitor, gelonin, mitogellin, restrictocin, phenomycin, neomycin, or trichothecene.

The radionuclide can be selected from the group consisting of ¹¹¹In, ⁹⁹Tc, ¹⁴C, ¹³¹I, ¹²⁵I, and 3H. The radioactive isotope can be selected from the group consisting of At²¹¹, I¹¹³, I¹²⁵, Y⁹⁰, Re¹⁸⁶, Re¹⁸⁸, Sm¹⁵³, Bi²¹², P³², Pb²¹², and Lu radioactive isotopes.

The chemotherapeutic drug can be selected from the group consisting of 5-fluorouracil, gemcitabine, maytansinoid, auristatin, dolastatin, calicheamicin or its derivatives, anthracycline, methotrexate, vindesine, paclitaxel, trichothecene, CC1065, vinca alkaloids, methotrexate, adriamycin, vincristine, vinblastine, etoposide, doxorubicin, melphalan, mitomycin C, chlorambucil, and daunorubicin.

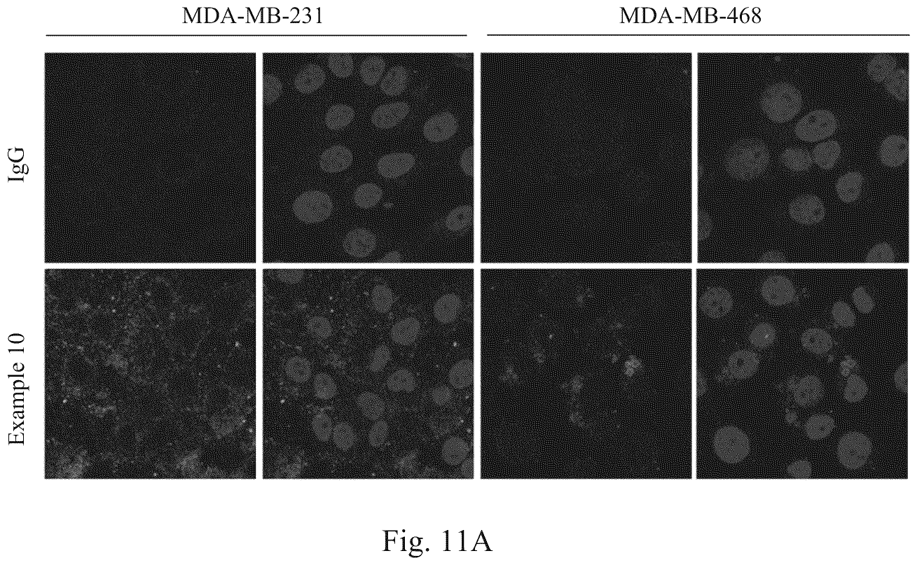

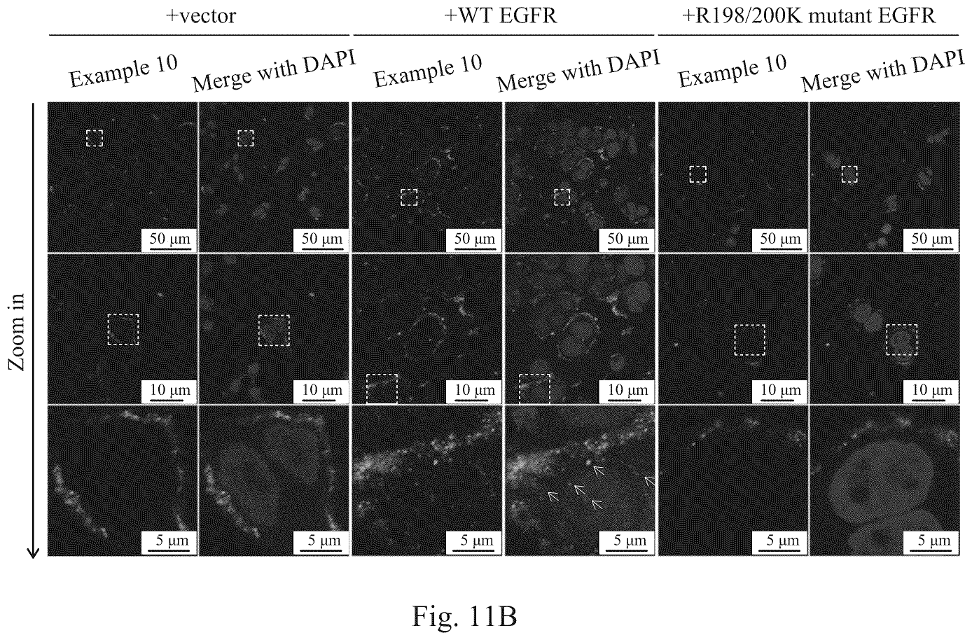

First, to test whether the antibody conjugate of the present disclosure can be internalized into tumor cells through endocytosis, Example 1 was conjugated with the dye DyLight 488 to form the antibody conjugate of Example 10 (hereinafter referred to as “Example 10”) as an example. Example 10 was then added to the culture medium of MDA-MB-231 and MDA-MB-468 and co-cultured for 30 minutes to observe whether Example 10 could be internalized into the cell of MDA-MB-231 and MDA-MB-468. Reference is made to FIG. 11A, which shows the analysis results of Example 10 being internalized by MDA-MB-231 and MDA-MB-468. The results show that green fluorescence signal (appears white in FIG. 11A) of Example 10 is observed in both MDA-MB-231 and MDA-MB-468, indicating that the TNBC cell lines MDA-MB-231 and MDA-MB-468 have good internalization ability for the antibody conjugate of the present disclosure.

In the experiment, the vector, the plasmid encoding wild-type EGFR, or the plasmid encoding R198/200K mutant EGFR (unmethylated mutant) were transfected into SW620. Example 10 was then added to the culture medium of the above cells and co-cultured for 30 minutes to observe whether Example 10 could be internalized into the cell of SW620.

Reference is made to FIG. 11B, which shows the analysis results of Example 10 being internalized by SW620. The results show that in SW620 transfected with the plasmid encoding wild-type EGFR, Example 10 was internalized into the cell of SW620. However, in SW620 transfected with the vector or the plasmid encoding R198/200K mutant EGFR (unmethylated mutant), the green fluorescence signal (appears white in FIG. 11B) of Example 10 is located around the cell membrane and is not internalized into the cell of SW620.

The above results indicate that the meEGFR antibody or the antigen-binding fragment thereof on the antibody conjugate of the present disclosure has high specificity for the meEGFR, thus the antibody conjugate of the present disclosure can recognize tumor cells expressing the meEGFR and be internalized into the tumor cells expressing the meEGFR through endocytosis. Therefore, the effector molecule on the antibody conjugate can be internalized into tumor cells along with the antibody conjugate, allowing the effector molecule to exert cytotoxic effect on the tumor cells.

To test whether the antibody conjugate of the present disclosure can induce tumor cell death, the anti-meEGFR antibody or the antigen-binding fragment thereof of the present disclosure was conjugated with the effector molecule to form another example of the antibody conjugate. In detail, Example 1 was conjugated with the chemotherapeutic drug monomethyl auristatin E (MMAE) to form the antibody conjugate of Example 11 (hereinafter referred to as “Example 11”). Based on cysteine, Example 11 can be constructed using both continuous conjugation method and in situ conjugation method. The linker-MMAE payload was synthesized using a peptide-based maleimide hexyl-L-valine-L-glutamine-para-aminophenylmethanol-nitrobenzenecarbamate (Mc-Val-Cit-PABC-PNP) linker or a rebridging linker reacting with MMAE.

The conventional linker-MMAE payload was constructed as follows. Dissolve and mix 18.20 μmol of MMAE, 16.38 μmol of Mc-Val-Cit-PABC-PNP, and 3.64 μmol of hydroxybenzotriazole in 500 μL of dimethylformamide. Add 18.20 μmol of pyridine to the mixture and mix frequently after 2 minutes. Subsequently, add 20 μmol of trifluoroacetic acid (TFA) and complete the reaction after 24 hours.

The method for constructing rebridging linker-MMAE payload was as follows. The rebridging linker was first synthesized by mixing 3.91 mmol of 6-aminohexanoic acid and 3.91 mmol of 3,4-dibromo-furan-2,5-dione in 20 mL acetic acid. After stirring at room temperature for 10 minutes, the solution was heated at 100 °C for 18 hours. The solvent was removed under vacuum, and the rebridging linker was purified by silica gel chromatography, using an elution of 0-40% dichloromethane/ethyl acetate. In 0.25 mL of dichloromethane, 13.55 μmol of N,N′-diisopropylcarbodiimide, 13.55 μmol of N,N-diisopropylethylamine, and 33.85 μmol of the rebridging linker were mixed and frequently stirred at room temperature for 1 hour. Then, 13.55 μmol of MMAE was added, and the mixture was frequently stirred for another 16 hours. The synthesized rebridging linker-MMAE was solvent removed via a vacuum pump and then purified using a Waters HPLC system equipped with a 600 controller/pump and a 996 PDA detector. The purification process utilized a reverse-phase C18 column (Phenomenex Luna) with 5 μm C18 (2) 100 Å and 250×10 mm, using gradient elution buffers of phase A (water+0.1% TFA) and phase B (acetonitrile). The purified product was confirmed using an Agilent 6500 series accurate-mass Q-TOF LC/MS (Agilent Technologies).

The continuous conjugation method was to reduce 5 mg/ml of the anti-meEGFR antibody or the antigen-binding fragment thereof with 1 mM dithiothreitol (DTT) in 50 mM borate buffer at pH 8.0 at 37 °C for 1 hour. Repeat buffer exchanges were performed using a Pierce dialysis column containing 1 mM PBS buffer. The conventional linker-MMAE payload and the rebridging linker-MMAE payload were mixed with the reduced anti-meEGFR antibody or the antigen-binding fragment thereof. The molar ratio of the conventional linker-MMAE payload to the anti-meEGFR antibody or the antigen-binding fragment thereof was 6.6:1, while the molar ratio for the rebridging linker-MMAE payload to the anti-meEGFR antibody or the antigen-binding fragment thereof was 4.4:1. After incubating at 4 °C for 1 hour, cysteamine in a molar excess of 20 times the payload was added to terminate the reaction. The final product was purified by G-25 gel filtration to obtain Example 11.

The in situ conjugation method was to reduce 5 mg/ml of the anti-meEGFR antibody or the antigen-binding fragment thereof using 7 equivalents of tris(2-carboxyethyl)phosphine (TCEP) in 50 mM borate buffer at pH 8.0. Simultaneously add the conventional linker-MMAE payload or the rebridging linker-MMAE payload and 7 equivalents of TCEP. After incubating at 37 °C for 2 hours, the product was purified by G-25 gel filtration to obtain Example 11.

The experiment further tested whether Example 11 can induce tumor cell death, and the tumor cells used were MDA-MB-468. The day before the experiment, MDA-MB-468 were seeded in a 96-well plate at a density of 8×10³ cells/well. After 16-18 hours of culture, the culture medium in the wells was removed, and DMEM-F12 medium containing different concentrations of Example 1, Example 11, or MMAE (concentrations of 0.1 nM, 1 nM, 10 nM, 100 nM, and 1000 nM respectively) was added, wherein the group adding MMAE was the positive control. After 48 hours of culture, the MTT assay was performed. The steps of the MTT assay were removing the culture medium from the 96-well plates, adding 100 μL of DMEM-F12 medium containing 5 μg/mL MTT, incubating in the incubator for 4 hours, completely removing the solution in the wells, adding 100 μL of DMSO to dissolve the crystals, and reading the absorbance at 570 nm using an enzyme immunoassay analyzer.

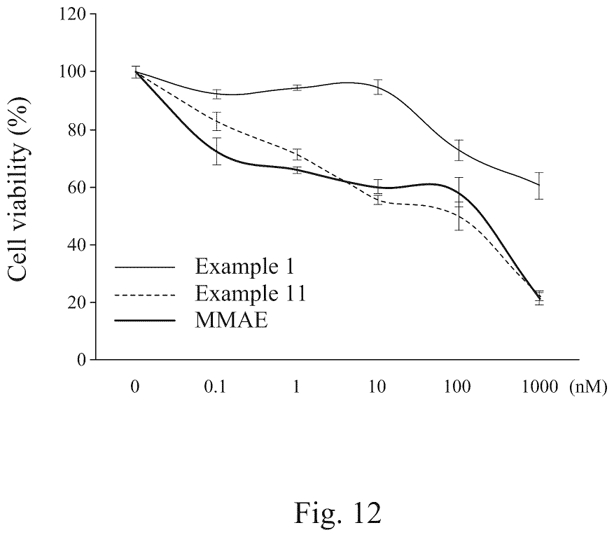

Reference is made to FIG. 12, which shows the analysis results of MDA-MB-468 cell death induced by Example 11. The results showed that Example 11 significantly induced MDA-MB-468 cell death, with a therapeutic effect superior to the group treated with the antibody (Example 1) alone, and the therapeutic effect of Example 11 was comparable to that of MMAE. Although MMAE has a good therapeutic effect, it cannot be used alone due to its toxicity. Therefore, MMAE can be conjugated with the anti-meEGFR antibody or the antigen-binding fragment thereof of the present disclosure to form the antibody conjugate, which can achieve the therapeutic effect comparable to that of MMAE while reducing the toxicity of MMAE thereof to the biological organism. Furthermore, the anti-meEGFR antibody or the antigen-binding fragment thereof of the present disclosure can be conjugated with other effector molecule, such as replacing MMAE with 5-fluorouracil or gemcitabine, which can not only reduce the toxicity of the chemotherapeutic drug to the biological organism but also further utilize the synergistic effect of the anti-meEGFR antibody or the antigen-binding fragment thereof of the present disclosure and the chemotherapeutic drug to achieve better therapeutic effect.

In summary, the present disclosure provides the anti-meEGFR antibody or the antigen-binding fragment thereof, the detection kit, the method for detecting the meEGFR-positive cancer, the pharmaceutical composition for treating the cancer, the meEGFR-specific chimeric antigen receptor, the nucleic acid encoding the meEGFR-specific chimeric antigen receptor, the meEGFR-specific chimeric antigen receptor expressing cell, the innate cell engager, and the antibody conjugate. The specification shows that the anti-meEGFR antibody or the antigen-binding fragment thereof of the present disclosure has high specificity and high affinity for the meEGFR, and shows that the anti-meEGFR antibody or the antigen-binding fragment thereof of the present disclosure is highly suitable for detecting the meEGFR in tumor tissues, and can be used to detect the meEGFR-positive cancer. The specification also shows that the pharmaceutical composition for treating the cancer including the anti-meEGFR antibody or the antigen-binding fragment thereof of the present disclosure has excellent inhibitory effect on growth of the tumor cell. Furthermore, the specification using tumor cell experiments shows that the meEGFR-specific chimeric antigen receptor and the expressing cell thereof developed based on the anti-meEGFR antibody or the antigen-binding fragment thereof of the present disclosure have excellent specific lysis ability for the tumor cell. The anti-meEGFR antibody or the antigen-binding fragment thereof of the present disclosure also has good internalization ability in the tumor cell. Therefore, the anti-meEGFR antibody or the antigen-binding fragment thereof of the present disclosure can be used as a basis to develop the antibody conjugate and the innate cell engager for clinical applications, overcoming and breaking through the current clinical difficulties in treating the meEGFR-positive cancer in many aspects, and providing a solution to the low response rate in cancer treatment that is resistant to conventional EGFR-targeted drug. Additionally, it can be applied to the treatment of various types of solid tumors in the meEGFR-positive cancers, significantly benefiting patients' medication choices and treatment outcomes. It has profound clinical significance, broad market value and global competitive advantages.

Although the present disclosure has been described as above by way of embodiments, it is not intended to limit the present disclosure. Person having skilled in the art can make various changes and modifications without departing from the spirit and scope of the present disclosure. Therefore, the scope of protection of the present disclosure should be defined by the scope of the appended patent application.

1. An anti-meEGFR antibody or an antigen-binding fragment thereof, which specifically binds to a methylated epidermal growth factor receptor (meEGFR) having asymmetric dimethylation modifications at R198 and R200 residues, wherein the anti-meEGFR antibody or the antigen-binding fragment thereof comprises:
a heavy chain variable region (V_{H}) comprising a heavy chain complementarity-determining region 1 (HCDR1), a heavy chain complementarity-determining region 2 (HCDR2), and a heavy chain complementarity-determining region 3 (HCDR3); and
a light chain variable region (V_{L}) comprising a light chain complementarity-determining region 1 (LCDR1), a light chain complementarity-determining region 2 (LCDR2), and a light chain complementarity-determining region 3 (LCDR3);
wherein the sequences of the HCDR1, the HCDR2, the HCDR3, the LCDR1, the LCDR2, and the LCDR3 are selected from the group consisting of:
the HCDR1 of SEQ ID NO: 7, the HCDR2 of SEQ ID NO: 8, the HCDR3 of SEQ ID NO: 9, the LCDR1 of SEQ ID NO: 10, the LCDR2 of SEQ ID NO: 11, and the LCDR3 of SEQ ID NO: 12;
the HCDR1 of SEQ ID NO: 13, the HCDR2 of SEQ ID NO: 14, the HCDR3 of SEQ ID NO: 15, the LCDR1 of SEQ ID NO: 16, the LCDR2 of SEQ ID NO: 17, and the LCDR3 of SEQ ID NO: 18; and
the HCDR1 of SEQ ID NO: 19, the HCDR2 of SEQ ID NO: 20, the HCDR3 of SEQ ID NO: 21, the LCDR1 of SEQ ID NO: 22, the LCDR2 of SEQ ID NO: 23, and the LCDR3 of SEQ ID NO: 24.

2. The anti-meEGFR antibody or the antigen-binding fragment thereof of claim 1, wherein the sequences of the V_{H} and the V_{L} are selected from the group consisting of:
the V_{H} comprising SEQ ID NO: 1 and the V_{L} comprising SEQ ID NO: 2;
the V_{H} comprising SEQ ID NO: 3 and the V_{L} comprising SEQ ID NO: 4; and
the V_{H} comprising SEQ ID NO: 5 and the V_{L} comprising SEQ ID NO: 6.

3. The anti-meEGFR antibody or the antigen-binding fragment thereof of claim 1, wherein the anti-meEGFR antibody or the antigen-binding fragment thereof is selected from the group consisting of a single-domain antibody, a humanized antibody, a multimeric antibody, a single-chain variable fragment (scFv), a Fab fragment, a Fab′ fragment, and a F(ab′)2 fragment.

4. A detection kit for detecting a meEGFR-positive cancer, comprising:
the anti-meEGFR antibody or the antigen-binding fragment thereof of claim 1.

5. The detection kit for detecting the meEGFR-positive cancer of claim 4, wherein the anti-meEGFR antibody or the antigen-binding fragment thereof is conjugated with a label, and the label is a fluorescent label, a chemiluminescent label, a radioactive isotope label, an enzyme label, a biotin label, or a combination thereof.

6. A method for detecting a meEGFR-positive cancer, comprising:
providing a test sample;
providing the detection kit for detecting the meEGFR-positive cancer of claim 4;
performing a binding step, wherein the test sample is contacted with the anti-meEGFR antibody or the antigen-binding fragment thereof to form a binding reaction; and
performing a detection step to detect whether the test sample comprises an antibody-cancer cell complex.

7. The method for detecting the meEGFR-positive cancer of claim 6, the meEGFR-positive cancer is a breast cancer, a colorectal cancer, a prostate cancer, a lung cancer, or a pancreatic cancer.

8. A pharmaceutical composition for treating a cancer, comprising:
the anti-meEGFR antibody or the antigen-binding fragment thereof of claim 1; and
a pharmaceutically acceptable carrier.

9. The pharmaceutical composition for treating the cancer of claim 8, further comprising a chemotherapeutic agent, an immunomodulator, a targeted therapeutic drug, an antibody drug, or a combination thereof, wherein the antibody drug is different from the anti-meEGFR antibody or the antigen-binding fragment thereof.

10. A meEGFR-specific chimeric antigen receptor, comprising:
an extracellular domain for recognizing a methylated epidermal growth factor receptor (meEGFR), and the meEGFR has asymmetric dimethylation modifications at R198 and R200 residues, wherein the extracellular domain comprises the anti-meEGFR antibody or the antigen-binding fragment thereof of claim 1; and
an intracellular signaling domain.

11. The meEGFR-specific chimeric antigen receptor of claim 10, further comprising a transmembrane domain linking the extracellular domain and the intracellular signaling domain.

12. The meEGFR-specific chimeric antigen receptor of claim 10, wherein the intracellular signaling domain comprises a co-stimulatory domain and a primary signaling domain.

13. A nucleic acid, which encodes the meEGFR-specific chimeric antigen receptor of claim 10.

14. A meEGFR-specific chimeric antigen receptor expressing cell, comprising:
an immune cell; and
the nucleic acid of claim 13;
wherein the meEGFR-specific chimeric antigen receptor expressing cell is obtained by transfecting the nucleic acid into the immune cell.

15. The meEGFR-specific chimeric antigen receptor expressing cell of claim 14, wherein the immune cell is a T cell or a natural killer cell.

16. A pharmaceutical composition for treating a cancer, comprising:
the meEGFR-specific chimeric antigen receptor expressing cell of claim 14; and
a pharmaceutically acceptable carrier.

17. An innate cell engager, comprising:
a meEGFR antigen recognition domain, comprising the anti-meEGFR antibody or the antigen-binding fragment thereof of claim 1; and
at least one immune cell receptor binding domain having a binding specificity for one or more of CD3, CD8, CD16, CD19, or NKG2D.

18. The innate cell engager of claim 17, wherein the innate cell engager is a bispecific T cell engager, a trispecific T cell engager, or a multispecific T cell engager.

19. The innate cell engager of claim 17, wherein the innate cell engager is a bispecific natural killer cell engager, a trispecific natural killer cell engager, or a multispecific natural killer cell engager.

20. An antibody conjugate, comprising:
the anti-meEGFR antibody or the antigen-binding fragment thereof of claim 1; and
an effector molecule, wherein the effector molecule is conjugated with the anti-meEGFR antibody or the antigen-binding fragment thereof through a chemical bond or a linker, and the effector molecule is a toxin, a growth inhibitor, a toxic protein, a radionuclide, a radioactive isotope, a chemotherapeutic drug, or a combination thereof.


SEQUENCE LISTING (67 sequences)

SEQ ID NO: 1 - protein, 473 aa, synthetic construct
MRAWIFFLLCLAGRALAAPLAQVQLVQSGAEVKKPGESLKISCKGSGYSFTSYWIGWVRQMPGKGLEWMGIIDPDDSETRYSPSFQGQVTISVDRSFSTAYLQWRSLAASDTAMYYCARSSSPYYDDLAFDIWGKGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 236 aa, synthetic construct
MRAWIFFLLCLAGRALAAPLAEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPITFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 3 - protein, 468 aa, synthetic construct
MRAWIFFLLCLAGRALAAPLAQVQLQESGPGLVKPSETLSLTCSVSGDSITRSGYYWAWVRQSPGKGLEYIGSISYSGTTYYNPSLKSRVTISVDTSKNQFSLKLSSVTAADTAVYYCAREWNGLDVWGKGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 238 aa, synthetic construct
MRAWIFFLLCLAGRALAAPLALNFMLTQPHSVSESPGKTVTISCTRSSGSIASNYVQWYQQRPGSSPTTVIYEDNQRPFGVPDRFSGSIDSSSNSASLTISGLKTEDEADYYCQSYDSGHQIFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 5 - protein, 468 aa, synthetic construct
MRAWIFFLLCLAGRALAAPLAQVQLQESGPGLVKPSGTLSLTCAVSGDSISSSSYYWGWIRQPPGKGLEWIGSIYYSGSTYYNPSLKSRVTISVDTSKNQFSLKLSSVTAADTAVYYCAREWNGLDVWGKGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 240 aa, synthetic construct
MRAWIFFLLCLAGRALAAPLALNFMLTQPHSVSESPGKTVTISCTGSSGSIASNYVQWYQQRPGSAPTTVIYEDNQRPSGVPDRFSGSIDSSSNSASLTISGLKTEDEADYYCQSYDRGNTEVMFGGGTKVTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 7 - protein, 5 aa, synthetic construct
SYWIG

SEQ ID NO: 8 - protein, 17 aa, synthetic construct
IIDPDDSETRYSPSFQG

SEQ ID NO: 9 - protein, 13 aa, synthetic construct
SSSPYYDDLAFDI

SEQ ID NO: 10 - protein, 12 aa, synthetic construct
RASQSVSSSYLA

SEQ ID NO: 11 - protein, 7 aa, synthetic construct
GASSRAT

SEQ ID NO: 12 - protein, 9 aa, synthetic construct
QQYGSSPIT

SEQ ID NO: 13 - protein, 7 aa, synthetic construct
RSGYYWA

SEQ ID NO: 14 - protein, 16 aa, synthetic construct
SISYSGTTYYNPSLKS

SEQ ID NO: 15 - protein, 7 aa, synthetic construct
EWNGLDV

SEQ ID NO: 16 - protein, 13 aa, synthetic construct
TRSSGSIASNYVQ

SEQ ID NO: 17 - protein, 7 aa, synthetic construct
EDNQRPF

SEQ ID NO: 18 - protein, 9 aa, synthetic construct
QSYDSGHQI

SEQ ID NO: 19 - protein, 7 aa, synthetic construct
SSSYYWG

SEQ ID NO: 20 - protein, 16 aa, synthetic construct
SIYYSGSTYYNPSLKS

SEQ ID NO: 21 - protein, 7 aa, synthetic construct
EWNGLDV

SEQ ID NO: 22 - protein, 13 aa, synthetic construct
TGSSGSIASNYVQ

SEQ ID NO: 23 - protein, 7 aa, synthetic construct
EDNQRPS

SEQ ID NO: 24 - protein, 11 aa, synthetic construct
QSYDRGNTEVM

SEQ ID NO: 25 - protein, 15 aa, synthetic construct
KQCSGRCRGKSPSDC

SEQ ID NO: 26 - protein, 15 aa, synthetic construct
  MOD_RES 6: note = asymmetric dimethylated
  MOD_RES 8: note = asymmetric dimethylated
KQCSGRCRGKSPSDC

SEQ ID NO: 27 - protein, 15 aa, synthetic construct
  MOD_RES 6: note = monomethyl
KQCSGRCRGKSPSDC

SEQ ID NO: 28 - protein, 15 aa, synthetic construct
  MOD_RES 8: note = monomethyl
KQCSGRCRGKSPSDC

SEQ ID NO: 29 - protein, 15 aa, synthetic construct
  MOD_RES 6: note = symmetric dimethyl
KQCSGRCRGKSPSDC

SEQ ID NO: 30 - protein, 15 aa, synthetic construct
  MOD_RES 8: note = symmetric dimethyl
KQCSGRCRGKSPSDC

SEQ ID NO: 31 - protein, 21 aa, synthetic construct
METDTLLLWVLLLWVPGSTGD

SEQ ID NO: 32 - protein, 246 aa, synthetic construct
QVQLVQSGAEVKKPGESLKISCKGSGYSFTSYWIGWVRQMPGKGLEWMGIIDPDDSETRYSPSFQGQVTISVDRSFSTAYLQWRSLAASDTAMYYCARSSSPYYDDLAFDIWGKGTLVTVSSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYGSSPITFGQGTRLEIKR

SEQ ID NO: 33 - protein, 29 aa, synthetic construct
IEVMYPPPYLDNEKSNGTIIHVKGKHLCP

SEQ ID NO: 34 - protein, 37 aa, synthetic construct
SPLFPGPSKPFWVLVVVGGVLACYSLLVTVAFIIFWV

SEQ ID NO: 35 - protein, 41 aa, synthetic construct
RSKRSRLLHSDYMNMTPRRPGPTRKHYQPYAPPRDFAAYRS

SEQ ID NO: 36 - protein, 104 aa, synthetic construct
APAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 37 - DNA, 63 bp, synthetic construct
  source 1..63: mol_type = other DNA
atggagacagacacactcctgctatgggtactgctgctctgggttccaggttccactggtgac

SEQ ID NO: 38 - DNA, 738 bp, synthetic construct
  source 1..738: mol_type = other DNA
caggtgcagctggtgcagtctggagcagaggtgaaaaagcccggggagtctctgaagatctcctgtaagggttctggatacagctttaccagctactggatcggctgggtgcgccagatgcccgggaaaggcctggagtggatggggatcatcgatcctgatgactctgaaaccagatacagcccgtccttccaaggccaggtcaccatctcagtcgacaggtccttcagtactgcctacctgcagtggaggagcctggcggcctcggacaccgccatgtattactgtgcgagatcctcctccccttattacgatgaccttgcttttgatatctggggcaaaggcaccctggtcaccgtctcgagtggtggaggcggttcaggcggaggtggttctggcggtggcggatcggaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacccatcaccttcggccaagggacacgactggagattaaacgt

SEQ ID NO: 39 - DNA, 87 bp, synthetic construct
  source 1..87: mol_type = other DNA
atcgaggtgatgtacccccccccctacctggacaacgagaagagcaacggcaccatcatccacgtgaagggcaagcacctgtgcccc

SEQ ID NO: 40 - DNA, 111 bp, synthetic construct
  source 1..111: mol_type = other DNA
agccccctgttccccggccccagcaagcccttctgggtgctggtggtggtgggcggcgtgctggcctgctacagcctgctggtgaccgtggccttcatcatcttctgggtg

SEQ ID NO: 41 - DNA, 123 bp, synthetic construct
  source 1..123: mol_type = other DNA
agaagcaagagaagcagactgctgcacagcgactacatgaacatgacccccagaagacccggccccaccagaaagcactaccagccctacgccccccccagagacttcgccgcctacagaagc

SEQ ID NO: 42 - DNA, 312 bp, synthetic construct
  source 1..312: mol_type = other DNA
gcccccgcctaccagcagggccagaaccagctgtacaacgagctgaacctgggcagaagagaggagtacgacgtgctggacaagagaagaggcagagaccccgagatgggcggcaagccccagagaagaaagaacccccaggagggcctgtacaacgagctgcagaaggacaagatggccgaggcctacagcgagatcggcatgaagggcgagagaagaagaggcaagggccacgacggcctgtaccagggcctgagcaccgccaccaaggacacctacgacgccctgcacatgcaggccctgccccccaga

SEQ ID NO: 43 - protein, 5 aa, synthetic construct
GGGGS

SEQ ID NO: 44 - DNA, 1419 bp, synthetic construct
  source 1..1419: mol_type = other DNA
atgagggcctggatcttctttctcctttgcctggccgggagggctctggcagccccgctagcacaggtgcagctggtgcagtctggagcagaggtgaaaaagcccggggagtctctgaagatctcctgtaagggttctggatacagctttaccagctactggatcggctgggtgcgccagatgcccgggaaaggcctggagtggatggggatcatcgatcctgatgactctgaaaccagatacagcccgtccttccaaggccaggtcaccatctcagtcgacaggtccttcagtactgcctacctgcagtggaggagcctggcggcctcggacaccgccatgtattactgtgcgagatcctcctccccttattacgatgaccttgcttttgatatctggggcaaaggcaccctggtcaccgtctcgagtgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 45 - DNA, 708 bp, synthetic construct
  source 1..708: mol_type = other DNA
atgagggcctggatcttctttctcctttgcctggccgggagggctctggcagccccgctagcagaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacccatcaccttcggccaagggacacgactggagattaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 46 - DNA, 1404 bp, synthetic construct
  source 1..1404: mol_type = other DNA
atgagggcctggatcttctttctcctttgcctggccgggagggctctggcagccccgctagcacaggtgcagctgcaggagtcgggcccaggactggtgaagccttcggagaccctgtccctcacctgtagtgtctctggtgactccatcaccaggagtggttactactgggcctgggtccgccagtccccagggaaggggctggaatacattgggagtatctcttacagtgggaccacctactacaacccgtccctcaagagtcgagtcaccatatcagtagacacgtccaagaaccagttctccctgaagctgagctctgtgaccgccgcggacacggctgtgtattactgtgcgagagagtggaacggtttggacgtctggggcaaagggacaatggtcaccgtctcgagtgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 47 - DNA, 714 bp, synthetic construct
  source 1..714: mol_type = other DNA
atgagggcctggatcttctttctcctttgcctggccgggagggctctggcagccccgctagcacttaattttatgctgactcagccccactctgtgtcggagtctccggggaagacggtaaccatctcctgcacccgcagcagtggcagcattgccagcaactatgtgcagtggtaccagcagcgcccgggcagttcccccaccactgtgatctatgaggataaccaaagaccttttggggtccctgatcgattctctggctccatcgacagctcctccaactctgcctccctcaccatctccggactgaagactgaggacgaggctgactactactgtcagtcttatgatagtggccatcagatattcggcggagggaccaagctgaccgtcctaggtcagcccaaggctgccccttccgtgaccctgtttcccccctcctccgaggagctgcaggccaacaaggccaccctggtgtgcctgatctccgacttctaccctggcgctgtgaccgtggcttggaaggccgattcctcccctgtgaaggccggcgtggagaccacaaccccctccaagcagtccaacaacaagtacgccgcttcctcctacctgtccctgacccccgagcagtggaagtcccacaggtcctactcctgccaggtgacccacgagggctccaccgtggagaagacagtggcccccaccgagtgctcc

SEQ ID NO: 48 - DNA, 1404 bp, synthetic construct
  source 1..1404: mol_type = other DNA
atgagggcctggatcttctttctcctttgcctggccgggagggctctggcagccccgctagcacaggtgcagctgcaggagtcgggcccaggactggtgaagccttcggggaccctgtccctcacctgcgctgtctctggtgactccatcagcagtagtagttactactggggctggatccgccagcccccagggaaggggctggagtggattgggagtatctattatagtgggagcacctactacaacccgtccctcaagagtcgagtcaccatatcagtagacacgtccaagaaccagttctccctgaagctgagctctgtgaccgccgcggacacggctgtgtattactgtgcgagagagtggaacggtttggacgtctggggcaaaggaaccctggtcaccgtctcgagtgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttcccaccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 49 - DNA, 720 bp, synthetic construct
  source 1..720: mol_type = other DNA
atgagggcctggatcttctttctcctttgcctggccgggagggctctggcagccccgctagcacttaattttatgctgactcagccccactctgtgtcggagtctccggggaagacggtaaccatctcctgcaccggcagcagtggcagcattgccagcaactatgtgcagtggtaccagcagcgcccgggcagtgcccccaccactgtgatctatgaggataaccaaagaccctctggggtccctgatcggttctctggctccatcgacagctcctccaactctgcctccctcaccatctctggactgaagactgaggacgaggctgactactactgtcagtcttatgatagaggcaataccgaggtgatgttcggcggagggaccaaggtcaccgtcctaggtcagcccaaggctgccccttccgtgaccctgtttcccccctcctccgaggagctgcaggccaacaaggccaccctggtgtgcctgatctccgacttctaccctggcgctgtgaccgtggcttggaaggccgattcctcccctgtgaaggccggcgtggagaccacaaccccctccaagcagtccaacaacaagtacgccgcttcctcctacctgtccctgacccccgagcagtggaagtcccacaggtcctactcctgccaggtgacccacgagggctccaccgtggagaagacagtggcccccaccgagtgctcc

SEQ ID NO: 50 - DNA, 15 bp, synthetic construct
  source 1..15: mol_type = other DNA
agctactggatcggc

SEQ ID NO: 51 - DNA, 51 bp, synthetic construct
  source 1..51: mol_type = other DNA
atcatcgatcctgatgactctgaaaccagatacagcccgtccttccaaggc

SEQ ID NO: 52 - DNA, 39 bp, synthetic construct
  source 1..39: mol_type = other DNA
tcctcctccccttattacgatgaccttgcttttgatatc

SEQ ID NO: 53 - DNA, 36 bp, synthetic construct
  source 1..36: mol_type = other DNA
agggccagtcagagtgttagcagcagctacttagcc

SEQ ID NO: 54 - DNA, 21 bp, synthetic construct
  source 1..21: mol_type = other DNA
ggtgcatccagcagggccact

SEQ ID NO: 55 - DNA, 27 bp, synthetic construct
  source 1..27: mol_type = other DNA
cagcagtatggtagctcacccatcacc

SEQ ID NO: 56 - DNA, 21 bp, synthetic construct
  source 1..21: mol_type = other DNA
aggagtggttactactgggcc

SEQ ID NO: 57 - DNA, 48 bp, synthetic construct
  source 1..48: mol_type = other DNA
agtatctcttacagtgggaccacctactacaacccgtccctcaagagt

SEQ ID NO: 58 - DNA, 21 bp, synthetic construct
  source 1..21: mol_type = other DNA
gagtggaacggtttggacgtc

SEQ ID NO: 59 - DNA, 39 bp, synthetic construct
  source 1..39: mol_type = other DNA
acccgcagcagtggcagcattgccagcaactatgtgcag

SEQ ID NO: 60 - DNA, 21 bp, synthetic construct
  source 1..21: mol_type = other DNA
gaggataaccaaagacctttt

SEQ ID NO: 61 - DNA, 27 bp, synthetic construct
  source 1..27: mol_type = other DNA
cagtcttatgatagtggccatcagata

SEQ ID NO: 62 - DNA, 21 bp, synthetic construct
  source 1..21: mol_type = other DNA
agtagtagttactactggggc

SEQ ID NO: 63 - DNA, 48 bp, synthetic construct
  source 1..48: mol_type = other DNA
agtatctattatagtgggagcacctactacaacccgtccctcaagagt

SEQ ID NO: 64 - DNA, 21 bp, synthetic construct
  source 1..21: mol_type = other DNA
gagtggaacggtttggacgtc

SEQ ID NO: 65 - DNA, 39 bp, synthetic construct
  source 1..39: mol_type = other DNA
accggcagcagtggcagcattgccagcaactatgtgcag

SEQ ID NO: 66 - DNA, 21 bp, synthetic construct
  source 1..21: mol_type = other DNA
gaggataaccaaagaccctct

SEQ ID NO: 67 - DNA, 33 bp, synthetic construct
  source 1..33: mol_type = other DNA
cagtcttatgatagaggcaataccgaggtgatg